(19) United States
(12) Patent Application Publication
(10) Pub. No.: US 2026/0000658 A1
(43) Pub. Date: Jan. 1, 2026

(54) METHODS FOR THE TREATMENT OF PROLIFERATIVE GLOMERULONEPHRITIS

(71) Applicants: INSTITUT NATIONAL DE LA SANTÉ ET DE LA RECHERCHE MÉDICALE, Paris (FR); UNIVERSITÉ PARIS CITÉ, Paris (FR); CENTRE NATIONAL DE LA RECHERCHE SCIENTIFIQUE, Paris (FR); ASSISTANCE PUBLIQUE-HÔPITAUX DE PARIS (APHP), Paris (FR)

(72) Inventors: Guillaume CANAUD, Paris (FR); Junna YAMAGUCHI, Paris (FR)

(21) Appl. No.: 18/881,047

(22) PCT Filed: Jul. 5, 2023
(86) PCT No.: PCT/EP2023/068553

§ 371 Date: Jan. 3, 2025

(30) Foreign Application Priority Data
Jul. 6, 2022 (EP) 22306011.2

(51) Int. Cl. A61K 31/4439; A61K 45/06; A61P 13/12

(52) U.S. Cl. CPC A61K 31/4439; A61K 45/06; A61P 13/12

(57) ABSTRACT

Inventors have explored the relevance of alpelisib in MRL/MpJ-Faslpr/J mice (referred here as MRL-lpr). a mouse model of lupus nephritis. MRL-lpr mice treated with alpelisib demonstrated less proteinuria compared to vehicle. More stinkingly, comparison of albuminuria before and after treatment introduction showed opposite trajectories. Indeed. MRL-lpr mice treated with alpelisib demonstrated proteinuria improvement arguing for a reversibility of the disease. At sacrifice. MRL-lpr mice treated with alpelisib had a tendency to have a lower kidney to body weight ratio compared to vehicle treated animals. Kidney examination showed that alpelisib was associated with no glomerular lesions compared to vehicle treated mice and an improved kidney function. Inventors conclude that alpelisib and more generally PIK3CA inhibition represent promising drugs for patients with proliferative glomerulonephritis. The invention relates to a method for treating proliferative glomerulonephritis in a subject in need thereof comprising a step of administering the subject with a therapeutically effective amount of a PIK3CA inhibitor.

### FIELD OF THE INVENTION

The invention relates to method and compositions for the treatment of proliferative glomerulonephritis, such as lupus nephritis or focal and segmental glomerulosclerosis.

### BACKGROUND OF THE INVENTION

PIK3CA is a ubiquitously expressed lipid kinase that controls signaling pathways participating in cell proliferation, motility, survival and metabolism¹. PIK3CA is mainly recruited through tyrosine kinase receptors. PIK3CA encodes the 110-kDa catalytic alpha subunit of PI3K (p110α), which converts, at the plasma membrane, phosphatidylinositol 4,5-bisphosphate (PtdIns(4,5)P2) to phosphatidylinositol 3,4,5-trisphosphate (PtdIns(3,4,5)P3; or PIP3) with subsequent recruitment of PDK1, which in turn phosphorylates AKT on the Thr308 residue to initiate downstream cellular effects. PIK3CA also regulates many other pathways, including the Rho/Rac1 signaling cascade².

Some subjects have a somatic gain of function mutation in the PIK3CA gene that leads to asymmetrical overgrowth called PIK3CA-Related Overgrowth Syndrome (PROS). PROS rare genetic disorders defined by tissue hypertrophy that can be either localized or generalized. The mutations are not inherited but occur during embryogenesis, conducting to somatic mosaicism³. The clinical presentation of patients with PIK3CA gain-of-function mutations is extremely broad owing to mosaicism but also the tissue involved⁴. Patients usually have complex tissue malformations, including abnormal vessels, anarchic adipose tissue, muscle hypertrophy and/or bone deformation⁵⁻⁹. Due to the wide variability of clinical presentation and the difficulty of genetic identification, which most often requires a biopsy of the affected area, the exact prevalence of PIK3CA gain-of-function mutations is yet unknown. We recently, created a mouse model that recapitulates PROS patient phenotype. We identified alpelisib, a PIK3CA inhibitor undergoing development in oncology as a promising therapeutic in the mouse model and were authorized to treat PROS patients in poor condition using this drug. Patients treated with alpelisib demonstrated clinical, biological and radiological improvements.

We previously reported that the first PROS patient treated with alpelisib had a kidney dysfunction with nephrotic range proteinuria. This chronic kidney disease was beside a complex medical situation with severe vascular malformations (mixed with vein and lymphatic anomalies but also arteriovenous shunts), paraplegia with vesicoureteral reflux, severe congestive heart failure and the use of rapamycin (an mTOR inhibitor). This drug is fairly well known to induce proteinuria in patients with chronic kidney disease (CKD)¹⁰. Rapamycin was stopped without clinical or biological changes.

Thus, there is a need to find new therapeutic strategy to treat proliferative glomerulonephritis.

### SUMMARY OF THE INVENTION

The present invention relates to a a method for treating proliferative glomerulonephritis in a subject in need thereof comprising a step of administrating the subject with a therapeutically effective amount of PI3K inhibitor, in particular PIK3CA inhibitor. In particular, the present invention is defined by the claims.

### DETAILED DESCRIPTION OF THE INVENTION

Inventors have explored the relevance of alpelisib in MRL/MpJ-Faslpr/J mice (referred here as MRL-lpr), a mouse model of lupus nephritis. MRL-lpr mice treated with alpelisib demonstrated less proteinuria compared to vehicle. More stinkingly, comparison of albuminuria before and after treatment introduction showed opposite trajectories. Indeed, MRL-lpr mice treated with alpelisib demonstrated proteinuria improvement arguing for a reversibility of the disease. At sacrifice, MRL-lpr mice treated with alpelisib had a tendency to have a lower kidney to body weight ratio compared to vehicle treated animals. Kidney examination showed that alpelisib was associated with no glomerular lesions compared to vehicle treated mice and an improved kidney function.

Inventors conclude that alpelisib and more generally PIK3CA inhibition represent promising drugs for patients with proliferative glomerulonephritis.

Accordingly, the present invention relates to a method for treating proliferative glomerulonephritis in a subject in need thereof comprising a step of administrating the subject with a therapeutically effective amount of PI3K inhibitor, in particular PIK3CA inhibitor.

In some embodiments, the present invention also relates to a method for treating proliferative glomerulonephritis in a subject in need thereof, wherein the method consists essentially in a step of administrating the subject with a therapeutically effective amount of PIK3CA inhibitor.

In some embodiments, the present invention also relates to a method for treating proliferative glomerulonephritis in a subject in need thereof, wherein the method consists in a step of administrating the subject with a therapeutically effective amount of PIK3CA inhibitor.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein the term “proliferative glomerulonephritis” (PGN) refers to an increase of cellularity of the glomerulus, which due to proliferation of intrinsic glomerular cells, infiltration of leucocytes, or both. This principally occurs in the context of glomerular deposition of immunoglobulins, immune complexes, or complement components. Different subtypes are described based on histological features: proliferation of mesangial cells, endocapillary proliferation, diffuse proliferation, or extracapillary proliferation (also termed crescentic glomerulonephritis). In some embodiments, the proliferative glomerulonephritis is extracapillary proliferative glomerulonephritis.

In a particular embodiment, the proliferative glomerulonephritis is caused by the following diseases selected from the group consisting of but not limited to: Infectious disease (poststreptococcal glomerulonephritis, infective endocarditis, occult visceral sepsis, hepatitis B infection—with vasculitis and/or cryoglobulinemia-, HIV infection, hepatitis C—with cryoglobulinemia, membranoproliferative glomerulonephritis-), multisysteme diseases (systemic lupus erythematosus, IgA nephropathy, Henoch-Schönlein purpura, systemic necrotizing vasculitis—including granulomatosis with polyangiitis type Wegener, Goodpasture's syndrome, essential mixed cryoglobulinemia, malignancy, relapsing polychondritis, rheumatoid arthritis—with vasculitis). In some embodiments, the vasculitis is an Anti-Neutrophil Cytoplasmic Autoantibody (ANCA) vasculitis.

In a particular embodiment, the proliferative glomerulonephritis is caused by systemic lupus erythematosus.

As used herein, the term “systemic lupus erythematosus” (SLE) refers to a systemic autoimmune disease thought to be manifested by a wide range of abnormalities in immune regulation. It is the most common type of lupus.

In a particular embodiment, the proliferative glomerulonephritis is lupus nephritis.

As used herein, the term “lupus nephritis” (LN) refers to an inflammation of the kidney that is caused by systemic lupus erythematosus (SLE). Up to 60% of lupus patients will develop LN. When the kidneys are inflamed, they cannot function normally to filter toxins, byproducts, excess salts, excess fluid, and other impurities the blood. If not controlled, LN can lead to kidney failure. Even with treatment, loss of kidney function sometimes progresses. If both kidneys fail, subjects with LN may need dialysis. Ultimately, it may be necessary for the LN subject to receive a kidney transplant. Symptoms of loss of or abnormal kidney function include increased amounts of protein in urine (proteinuria), foaming in a subject's urine, and/or a higher level of blood urea nitrogen (BUN). In another particular embodiment, the proliferative glomerulonephritis is Focal and Segmental Glomerulosclerosis (FSGS).

As used herein, the term “subject” refers to any mammals, such as a rodent, a feline, a canine, and a primate. Particularly, in the present invention, the subject is a human afflicted with or susceptible to be afflicted with at least one of disorder proliferative glomerulonephritis as described above.

In a particular embodiment, the subject is a human afflicted with or susceptible to be afflicted with infectious disease (poststreptococcal glomerulonephritis, infective endocarditis, occult visceral sepsis, hepatitis B infection-with vasculitis and/or cryoglobulinemia-, HIV infection, hepatitis C—with cryoglobulinemia, membranoproliferative glomerulonephritis-) or multisysteme diseases (systemic lupus erythematosus, IgA nephropathy, Henoch-Schönlein purpura, systemic necrotizing vasculitis-including granulomatosis with polyangiitis type Wegener, Goodpasture's syndrome, essential mixed cryoglobulinemia, malignancy, relapsing polychondritis, rheumatoid arthritis-with vasculitis). In some embodiments, the vasculitis is an Anti-Neutrophil Cytoplasmic Autoantibody (ANCA) vasculitis.

In another embodiment, the subject is a human afflicted with or susceptible to be afflicted with proliferative glomerulonephritis.

In another embodiment, the subject is a human afflicted with or susceptible to be afflicted with endo and extra capillary proliferative glomerulonephritis.

In another embodiment, the subject is a human afflicted with or susceptible to be afflicted with mesangial proliferative glomerulonephritis.

In another embodiment, the subject is a human afflicted with or susceptible to be afflicted with diffuse capillary proliferative glomerulonephritis.

In another embodiment, the subject is a human afflicted with or susceptible to be afflicted with systemic lupus erythematosus (SLE).

In another embodiment, the subject is a human afflicted with or susceptible to be afflicted with lupus nephritis (LN).

In some embodiment, the subject is a human afflicted with or susceptible to be afflicted with Focal and Segmental Glomerulosclerosis (FSGS).

As used herein, the term “PI3K” refers to phosphoinositide 3-kinases also called phophatidylinositide 3-kinases. PI3K belongs to a family of enzymes which phosphorylate the 3′hydroxyl group of the onositol ring of the phosphatidylinositol (PtdIns). The PI3K signalling pathway can be activated, resulting in the synthesis of PIP3 from PIP2. PIK3CA is mainly recruited through tyrosine kinase receptors. PIK3CA encodes the 110-kDa catalytic alpha subunit of PI3K (p110α), which converts, at the plasma membrane, phosphatidylinositol 4,5-bisphosphate (PtdIns(4,5)P2) to phosphatidylinositol 3,4,5-trisphosphate (PtdIns(3,4,5)P3; or PIP3) with subsequent recruitment of PDK1, which in turn phosphorylates AKT on the Thr308 residue to initiate downstream cellular effects. PIK3CA also regulates many other pathways, including the Rho/Rac1 signaling cascade.

As used herein, the term “PI3K inhibitor” refers to a natural or synthetic compound that has a biological effect to inhibit the activity or the expression of PI3K. More particularly, such compound is capable of inhibiting the kinase activity of at least one member of PI3K family, for example, at least a member of Class I PI3K. In particular embodiment, said PI3K inhibitor may be a pan-inhibitor of Class I PI3K (known as p110) or isoform specific of Class I PI3K isoforms (among the four types of isoforms, p110α, p110β, p110γ or p110δ).

In a particular embodiment, the PI3K inhibitor is a peptide, peptidomimetic, small organic molecule, antibody, aptamers, siRNA or antisense oligonucleotide. The term “peptidomimetic” refers to a small protein-like chain designed to mimic a peptide. In a particular embodiment, the inhibitor of PI3K is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, the PI3K inhibitor is a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In a particular embodiment, the PI3K inhibitor is a small molecule which is an isoform-selective inhibitor of PI3K selected among the following compounds: BYL719 (Alpelisib, Novartis), GDC-0032 (Taselisib, Genentech/Roche), BKM120 (Buparlisib), A66 (University of Auckland), GDC0941 (Pictilisib, Genentech), PX-866 (Oncothyreon), Dactolisib, Voxtalisib (SAR245409, XL765), Pilaralisib, GDC-0077 (inavolisib, Genentech/Roche), CYH33 (risovalisib), TAK-117/MLN1117/INK1117 (serabelisib), BAY80-6946 (Copanlisib, Bayer Healthcare) or their pharmaceutically acceptable salts. In a more particular embodiment, the isoform-selective inhibitor of PI3K is selected among the following compounds: BYL719 (Alpelisib, Novartis), A66 (University of Auckland), GDC-0077 (inavolisib, Genentech/Roche), CYH33 (risovalisib), TAK-117/MLN1117/INK1117 (serabelisib) or their pharmaceutically acceptable salts. In an even more particular embodiment, the isoform-selective inhibitor of PI3K is selected among the following compounds: BYL719 (Alpelisib, Novartis), GDC-0077 (inavolisib, Genentech/Roche), TAK-117/MLN1117/INK1117 (serabelisib) or their pharmaceutically acceptable salts. Such PI3K inhibitors are well-known in the art and described for example in Wang et al Acta Pharmacological Sinica (2015) 36:1170-1176.

In a particular embodiment, the PI3K inhibitor is BYL719 and its derivatives. As used herein, the term “BYL719” also called alpelisib is an ATP-competitive oral PI3K inhibitor selective for the p110α isoform that is activated by a mutant PIK3CA gene (Furet P., et al. 2013; Fritsch C., et al 2014). This molecule is also called Alpelisib and has the following formula and structure in the art C₁₉H₂₂F₃N₅O₂S:

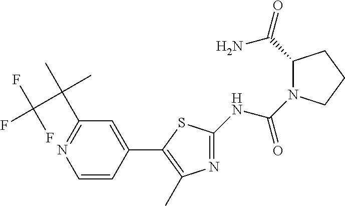 [SMILES: Cc1nc(NC(=O)N2CCC[C@H]2C(N)=O)sc1-c1ccnc(C(C)(C)C(F)(F)F)c1]

In a particular embodiment, the PI3K inhibitor is GDC-0032 and its derivatives, developed by Roche. This molecule also called Taselisib has the following formula and structure in the art C₂₄H₂₈N₈O₂:

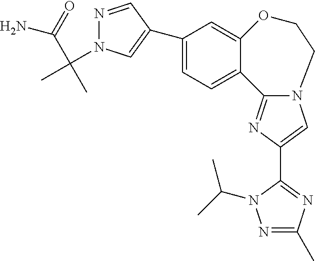 [SMILES: Cc1nc(-c2cn3c(n2)-c2ccc(-c4cnn(C(C)(C)C(N)=O)c4)cc2OCC3)n(C(C)C)n1]

In some embodiments, the PI3K inhibitor is an antibody. As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa (lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/11161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388. In a particular embodiment, the inhibitor is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular, the PI3K inhibitor is an intrabody having specificity for PI3K. As used herein, the term “intrabody” generally refer to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In some embodiments, the according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb.

In some embodiments, the PI3K inhibitor is a short hairpin RNA (shRNA), a small interfering RNA (siRNA) or an antisense oligonucleotide which inhibits the expression of USP14. In a particular embodiment, the inhibitor of USP14 expression is siRNA. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene. Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the inhibitor of PI3K expression is an endonuclease. In the last few years, staggering advances in sequencing technologies have provided an unprecedentedly detailed overview of the multiple genetic aberrations in cancer. By considerably expanding the list of new potential oncogenes and tumor suppressor genes, these new data strongly emphasize the need of fast and reliable strategies to characterize the normal and pathological function of these genes and assess their role, in particular as driving factors during oncogenesis. As an alternative to more conventional approaches, such as cDNA overexpression or downregulation by RNA interference, the new technologies provide the means to recreate the actual mutations observed in cancer through direct manipulation of the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339:823-826), bacteria (Fabre et al., 2014, PLoS Negl. Trop. Dis., Vol. 8: e2671.), zebrafish (Hwang et al., 2013, PLoS One, Vol. 8: e68708.), C. elegans (Hai et al., 2014 Cell Res. doi: 10.1038/cr.2014.11.), bacteria (Fabre et al., 2014, PLoS Negl. Trop. Dis., Vol. 8: e2671.), plants (Mali et al., 2013, Science, Vol. 339:823-826), Xenopus tropicalis (Guo et al., 2014, Development, Vol. 141:707-714.), yeast (DiCarlo et al., 2013, Nucleic Acids Res., Vol. 41:4336-4343.), Drosophila (Gratz et al., 2014 Genetics, doi:10.1534/genetics.113.160713), monkeys (Niu et al., 2014, Cell, Vol. 156:836-843.), rabbits (Yang et al., 2014, J. Mol. Cell Biol., Vol. 6:97-99.), pigs (Hai et al., 2014, Cell Res. doi: 10.1038/cr.2014.11.), rats (Ma et al., 2014, Cell Res., Vol. 24:122-125.) and mice (Mashiko et al., 2014, Dev. Growth Differ. Vol. 56:122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13). In another embodiment, the invention relates to the PI3K inhibitor for use according to the invention, and a classical treatment as a combined preparation for simultaneous, separate or sequential use in the treatment of proliferative glomerulonephritis in a subject in need thereof.

As used herein, the terms “combined treatment”, “combined therapy” or “therapy combination” refer to a treatment that uses more than one medication. The combined therapy may be dual therapy or bi-therapy.

As used herein, the term “classical treatment” refers to treatments well known in the art and used to treat proliferative glomerulonephritis (Hahn et al 2013, Arthritis Care Res (Hoboken). 2012 June; 64(6): 797-808; doi: 10.1002/acr.21664).

In the context of the invention, the classical treatment is selected from the group consisting of but not limited to: immunosuppressor, glucocorticoid, MAPK, PAK, mTOR, TKI, PARP and/or EGFR inhibitors. When several inhibitors are used, a mixture of inhibitors is obtained. In the case of multi-therapy (for example, bi-, tri- or quadritherapy), at least one other inhibitor can accompany the PI3K inhibitor.

In a particular embodiment, the PI3K inhibitor as described above is combined with an immunosuppressive therapy.

As used herein, the term “immunosuppressive therapy” refers to immunosuppressive treatment, which means that the subject is administered with one or more immunosuppressive drugs. Immunosuppressive drugs that may be employed in transplantation procedures include azathioprine (AZA), methotrexate, cyclophosphamide (CYC), FK-506 (tacrolimus), rapamycin, corticosteroids, and cyclosporins. These drugs may be used in monotherapy or in combination therapies.

In a particular embodiment, the immunosuppressive treatment is performed with azathioprine.

In a particular embodiment, the immunosuppressive treatment is performed with cyclophosphamide.

In another embodiment, the PI3K inhibitor as described above is combined with glucocorticoids therapy.

As used herein, the term “glucocorticoids therapy” refers to a class of corticosteroids, which are a class of steroid hormones. Glucocorticoids are corticosteroids that bind to the glucocorticoid receptor.

In a particular embodiment, the glucocorticoid therapy is performed with prednisone.

In another embodiment, the classical treatment is mycophenolate mofetil (MMF, CELLCEPT).

In a particular embodiment, the PI3K inhibitor, an immunosuppressor and a glucocorticoid can be combined as a tri-therapy for use in the treatment of proliferative glomerulonephritis.

In a particular embodiment, the PI3K inhibitor, an immunosuppressor and a glucocorticoid can be combined as a tri-therapy, wherein the PI3K inhibitor, immunosuppressor and a glucocorticoid are BYL719, azathioprine or clophosphamide and prednisone respectfully.

In a particular embodiment, the PI3K inhibitor for use according to the invention, and an immunosuppressor, glucocorticoids, MAPK, PAK, mTOR, TK, PARP or EGFR inhibitor as a combined preparation for simultaneous, separate or sequential use in the treatment of proliferative glomerulonephritis in a subject in need thereof.

In a particular embodiment, the PI3K inhibitor for use according to the invention, and an immunosuppressor, glucocorticoids, MAPK, PAK, mTOR, TK, PARP or EGFR inhibitor a combined preparation for simultaneous, separate or sequential use in the treatment of lupus nephritis in a subject in need thereof.

In a particular embodiment, the PI3K inhibitor for use according to the invention, and an immunosuppressor, glucocorticoids, MAPK, PAK, mTOR, TK, PARP or EGFR inhibitor as a combined preparation for simultaneous, separate or sequential use in the treatment of Focal and Segmental Glomerulosclerosis (FSGS) in a subject in need thereof.

In another embodiment, the invention relates to a combination comprising a PI3K inhibitor, and at least one classical treatment selected from the group consisting of immunosuppressor, glucocorticoids, MAPK, PAK, mTOR, TK, PARP or EGFR inhibitors as described below for use in the treatment of proliferative glomerulonephritis in a subject in need thereof.

In another embodiment, the PI3K, MAPK and PAK inhibitors can be combined as a tri-therapy for use in the treatment of proliferative glomerulonephritis. In a particular embodiment, the PI3K, MAPK and PAK inhibitors can be combined as a tri-therapy, wherein the PI3K, MAPK and inhibitors are BYL719, selumetinib and IPA-3 respectfully. The present invention also relates to a method for treating proliferative glomerulonephritis in a subject in need thereof comprising a step of administering the subject with a therapeutically effective amount of a PI3K inhibitor. In a particular embodiment, the method according to the invention, wherein the PI3K inhibitor and a MAPK inhibitor, a PAK inhibitor, an mTOR inhibitor, a TKI, a PARP inhbitor or an EGFR inhibitor, as combined preparation for use simultaneously, separately or sequentially in the treatment of proliferative glomerulonephritis.

As used herein, the term “MAPK” refers to mitogen-activated protein kinase, is a type of protein kinase that is specific to the amino acids serine and threonine. MAPK are involved in cellular responses to a diverse array of stimuli, such as mitogens, osmotic stress, heat shock and proinflammatory cytokines. Six groups of MAPK have so far been identified: Extracellular signal-regulated kinases (ERK1, ERK2), c-Jun N-terminal kinases (JNKs), p38 isoforms (MAPK11, MAPK12, MAPK13, MAPK14), ERK5 (MAPK7), ERK3 (MAPK6) and ERK4 (MAPK4), ERK7/8 (MAPK15). In a particular embodiment, the inhibitors of MAPK are inhibitors of ERKI/ERK2. The inhibitor of ERK1/ERK2 is selected from the group but is not limited to VTX-11e, SCH772984.

In a particular embodiment, the MAPK inhibitor is a peptide, peptidomimetic, small organic molecule, antibody, aptamers, siRNA or antisense oligonucleotide. In a particular embodiment, the MAPK inhibitor is p38-MAPK inhibitor. Typically, the inhibitor of p38-MAPK is selected from the group consisting of SB 203580, SB 203580 hydrochloride, SB681323 (Dilmapimod), LY2228820 dimesylate, BIRB 796 (Doramapimod), BMS-582949, Pamapimod, GW856553, ARRY-797AL 8697, AMG 548, CMPD-1, EO 1428, JX 401, RWJ 67657, TA 01, TA 02, VX 745,DBM 1285 dihydrochloride, ML 3403, SB 202190, SB 239063, SB 706504, SCIO 469 hydrochloride, SKF 86002 dihydrochloride, SX 011, TAK 715, VX 702, or PH-797804.

In a particular embodiment, the inhibitor of MAPK is an inhibitor of MEK. MEK1 and MEK2 are members of a larger family of dual-specificity kinases (MEK1-7) that phosphorylate threonine and tyrosine residues of various MAP kinases. In a particular embodiment, the inhibitor of MAPK is selected from the group consisting of Trametinib (GSK1120212); Selumetinib (AZD6244).

In a particular embodiment, the PI3K inhibitor for use according to the invention and, a MAPK inhibitor, as a combined preparation for simultaneous, separate or sequential use in the treatment of proliferative glomerulonephritis in a subject in need thereof, wherein the PI3K inhibitor is BYL719 and, the MAPK inhibitor is Selumetinib.

In another embodiment, the PI3K inhibitor for use according to the invention, and PAK inhibitor as a combined preparation for simultaneous, separate or sequential use in the treatment of proliferative glomerulonephritis in a subject in need thereof.

As used herein, the term “PAK” refers to p21-activated kinase which regulates cytoskeleton remodeling, phenotypic signaling and gene expression, and affects a wide variety of cellular processes such as directional motility, invasion, metastasis, growth, cell cycle progression, angiogenesis. In a particular embodiment, the PAK inhibitor is a peptide, peptidomimetic, small organic molecule, antibody, aptamers, siRNA or antisense oligonucleotide.

In a particular embodiment, the inhibitor of PAK is selected from the group consisting of PP1, hPIP1, NESH, Merlin, CRIPak, LKB1, Mesalamine, Glaucarubinone, Myricetin, β-elemene, miR-7, miR-let-7, miR-145, FRAX1036, OSU-03012, and IPA-3.

In a particular embodiment, the PAK inhibitor is used with thalidomide, lenalidomide or pomalidomide, as a combined preparation for use in the treatment of proliferative glomerulonephritis.

In a particular embodiment, the PI3K inhibitor for use according to the invention and, a PAK inhibitor, as a combined preparation for simultaneous, separate or sequential use in the treatment of proliferative glomerulonephritis in a subject in need thereof, wherein the PI3K inhibitor is BYL719 and the PAK inhibitor is IPA-3.

In another embodiment, the PI3K inhibitor for use according to the invention, and mTOR inhibitor as a combined preparation for simultaneous, separate or sequential use in the treatment of proliferative glomerulonephritis in a subject in need thereof.

As used herein, the term “mTOR” refers to mammalian target of rapamycin also known as mechanistic target of rapamycin and FK506-binding protein 12-rapamycin-associated protein 1 (FRAP1). mTOR functions as a serine/threonine protein kinase that regulates cell growth, cell proliferation, cell motility, cell survival, protein synthesis, autophagy, and transcription. mTOR has two structurally distinct complexes: mTORC1 and mTORC2. In a particular embodiment, the mTOR inhibitor is a peptide, peptidomimetic, small organic molecule, antibody, aptamers, siRNA or antisense oligonucleotide.

In a particular embodiment, the inhibitor of mTOR is selected from the group consisting of rapamycin (also called sirolimus and described in U.S. Pat. No. 3,929,992), temsirolimus, deforolimus, everolimus, tacrolimus and rapamycin analogue or derivative thereof, AMG954, AZD8055, AZD2014, BEZ235, BGT226, CC-115, CC-223, LY3023414, P7170, DS-7423, OSI-027, GSK2126458, PF-04691502, PF-05212384, INK128, MLN0128, MLN1117, Ridaforolimus, Metformin, XL765, SAR245409, SF1126, VS5584, GDC0980 and GSK2126458.

As used herein, the term “rapamycin analogue or derivative thereof” includes compounds having the rapamycin core structure as defined in U.S. Patent Application Publication No. 2003/0008923 (which is herein incorporated by reference), which may be chemically or biologically modified while still retaining mTOR inhibiting properties. Such derivatives include esters, ethers, oximes, hydrazones, and hydroxylamines of rapamycin, as well as compounds in which functional groups on the rapamycin core structure have been modified, for example, by reduction or oxidation. Pharmaceutically acceptable salts of such compounds are also considered to be rapamycin derivatives. Specific examples of esters and ethers of rapamycin are esters and ethers of the hydroxyl groups at the 42- and/or 31-positions of the rapamycin nucleus, and esters and ethers of a hydroxyl group at the 27-position (following chemical reduction of the 27-ketone). Specific examples of oximes, hydrazones, and hydroxylamines are of a ketone at the 42-position (following oxidation of the 42-hydroxyl group) and of 27-ketone of the rapamycin nucleus.

Examples of 42- and/or 31-esters and ethers of rapamycin are disclosed in the following patents, which are hereby incorporated by reference in their entireties: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); acetals (U.S. Pat. No. 551,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl,-alkyl,-alkenyl, and- alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462).

Examples of 27-esters and ethers of rapamycin are disclosed in U.S. Pat. No. 5,256,790, which is hereby incorporated by reference in its entirety.

Examples of oximes, hydrazones, and hydroxylamines of rapamycin are disclosed in U.S. Pat. Nos. 5,373,014, 5,378,836, 5,023,264, and 5,563,145, which are hereby incorporated by reference. The preparation of these oximes, hydrazones, and hydroxylamines is disclosed in the above listed patents. The preparation of 42-oxorapamycin is disclosed in U.S. Pat. No. 5,023,263, which is hereby incorporated by reference.

Other compounds within the scope of “rapamycin analog or derivative thereof” include those compounds and classes of compounds referred to as “rapalogs” in, for example, WO 98/02441 and references cited therein, and “epirapalogs” in, for example, WO 01/14387 and references cited therein.

Another compound within the scope of “rapamycin derivatives” is everolimus, a 4-O-(2-hydroxyethyl)-rapamycin derived from a macrolide antibiotic produced by Streptomyces hygroscopicus (Novartis). Everolimus is also known as Certican, RAD-001 and SDZ-RAD. Another preferred mTOR inhibitor is zotarolimus, an antiproliferative agent (Abbott Laboratories). Zotarolimus is believed to inhibit smooth muscle cell proliferation with a cytostatic effect resulting from the inhibition of mTOR. Another preferred mTOR inhibitor is tacrolimus, a macrolide lactone immunosuppressant isolated from the soil fungus Streptomyces tsukubaensis. Tacrolimus is also known as FK 506, FR 900506, Fujimycin, L 679934, Tsukubaenolide, PROTOPIC and PROGRAF. Other preferred mTOR inhibitors include AP-23675, AP-23573, and AP-23841 (Ariad Pharmaceuticals).

Preferred rapamycin derivatives include everolimus, CCI-779 (rapamycin 42-ester with 3-hydroxy-2-(hydroxymethyl)-2-methylpropionic acid; U.S. Pat. No. 5,362,718); 7-epi-rapamycin; 7-thiomethyl-rapamycin; 7-epi-trimethoxyphenyl-rapamycin; 7-epi-thiomethyl-rapamycin; 7-demethoxy-rapamycin; 32-demethoxy-rapamycin; 2-desmethyl-rapamycin; and 42-O-(2-hydroxy) ethyl rapamycin (U.S. Pat. No. 5,665,772).

Additional mTORC2 inhibitors may be OSI-027 (OSI Pharmaceuticals), a small molecule mTORC2 inhibitor. OSI-027 inhibits mTORC2 signaling complexes, allowing for the potential for complete truncation of aberrant cell signaling through this pathway.

In addition, torkinibs, ATP-competitive mTOR kinase domain inhibitors and inhibitors of mTORC2 may also be used according to the invention. Exemplary torkinibs include PP242 and PP30 (see, Feldman et al. (2009) PLoS Biology 7:371) and Torin1 (Thoreen et al. (2009) J Biol Chem 284:8023).

In a particular embodiment, the PI3K inhibitor for use according to the invention and, a mTOR inhibitor, as a combined preparation for simultaneous, separate or sequential use in the treatment of proliferative glomerulonephritis in a subject in need thereof, wherein the PI3K inhibitor is BYL719 and the mTOR inhibitor is everolimus.

In another embodiment, the PI3K inhibitor for use according to the invention, and tyrosine kinase inhibitor (TKI) as a combined preparation for simultaneous, separate or sequential use in the treatment of proliferative glomerulonephritis in a subject in need thereof.

As used herein, the term “TKI” refers to tyrosine kinase inhibitor. Tyrosine kinase is involved in the phosphorylation of many proteins. Example of tyrosine kinase proteins: AATK; ABL; ABL2; ALK; AXL; BLK; BMX; BTK; CSF1R; CSK; DDR1; DDR2; EGFR; EPHA1; EPHA2; EPHA3; EPHA4; EPHA5; EPHA6; EPHA7; EPHA8; EPHA10; EPHB1; EPHB2; EPHB3; EPHB4; EPHB6; ERBB2; ERBB3; ERBB4; FER; FES; FGFR1; FGFR2; FGFR3; FGFR4; FGR; FLT1; FLT3; FLT4; FRK; FYN; GSG2; HCK; IGF1R; ILK; INSR; INSRR; IRAK4; ITK; JAK1; JAK2; JAK3; KDR; KIT; KSR1; LCK; LMTK2; LMTK3; LTK; LYN; MATK; MERTK; MET; MLTK; MST1R; MUSK; NPR1; NTRK1; NTRK2; NTRK3; PDGFRA; PDGFRB; PLK4; PTK2; PTK2B; PTK6; PTK7; RET; ROR1; ROR2; ROS1; RYK; SGK493; SRC; SRMS; STYK1; SYK; TEC; TEK; TEX14; TIE1; TNK1; TNK2; TNNI3K; TXK; TYK2; TYRO3; YES1; ZAP70. In a particular embodiment, the TKI is a peptide, peptidomimetic, small organic molecule, antibody, aptamers, siRNA or antisense oligonucleotide.

In a particular embodiment, the tyrosine kinase is EGFR. As used herein, the term “EGFR” refers to epidermal growth factor receptor which is a member of the ErbB family of receptors, a subfamily of four closely related receptor tyrosine kinases: EGFR (ErbB-1), HER2/neu (ErbB-2), Her 3 (ErbB-3) and Her 4 (ErbB-4). EGFR are involved in the differentiation and cell growth. Inhibitors of EGFR refer to compounds which inhibits cell growth. In a particular embodiment, the inhibitor of EGFR is selected from the group consisting of: gefitinib, erlotinib, afatinib, brigatinib, lapatinib, icotinib, cetuximab Osimertinib, zalutumumab, nimotuzumab, and matuzumab.

In a particular embodiment, the inhibitor of EGFR is an irreversible mutant-selective EGFR inhibitor that specifically targets EGFR-activating mutations arising de novo and upon resistance acquisition. Typically, such inhibitor inhibits the most common EGFR mutations L858R, Ex19del, and T790M. Accordingly, in a particular embodiment, the inhibitor of EGFR is EGF816 also known as Nazartinib developed by Novartis.

In a particular embodiment, the tyrosine kinase is VEGF. As used herein, the term “VEGF” refers to vascular endothelial growth factor. VEGF is involved in stimulate cellular responses by binding to tyrosine kinase receptors (the VEGFRs) on the cell surface, notably to stimulate the formation of blood vessel (angiogenesis). VEGF family comprises in mammals five members: VEGF-A, placenta growth factor (PGF), VEGF-B, VEGF-C and VEGF-D. In a particular embodiment, the inhibitors of VEGF refer to inhibit the stimulation of growth cells and formation of blood vessel. In a particular embodiment, the inhibitor of VEGF is selected from the group consisting of: ranibizumab (Lucentis®), aflibercept (Eylea®) and bevacizumab (Avastin®), Tivozanib, Lenvatinib, Axitinib, Imtinib, or brolucizumab (RTH258).

In another embodiment, the inhibitor is a VEGFR inhibitor. As used herein, the term “VEGFR” refers to receptors for vascular endothelial growth factor (VEGF). Three main subtypes of VEGFR exist: VEGFRI, VEGFR 2 and VEGFR 3. VEGFR inhibitor is selected from the group consisting of: Pegaptanib, lenvatinib, motesanib, Pazopanib, cabozantinib (cabometyx®).

In some embodiments, the TKI is selected from the group consisting of gefitinib, erlotinib, dasatinib, nilotinib, bosutinib, ponatinib, ruxolitinib, quizartinib, cabozantinib and sunitinib. In a specific embodiment, the TKI is imatinib.

In another embodiment, the PI3K inhibitor for use according to the invention, and PARP inhibitor as a combined preparation for simultaneous, separate or sequential use in the treatment of proliferative glomerulonephritis in a subject in need thereof.

As used herein, the term “PARP” refers to Poly (ADP-ribose) polymerase which is an enzyme involved in cellular processes such as DNA repair, genomic stability, and programmed cell death. In a particular embodiment, the PARP inhibitor is a peptide, peptidomimetic, small organic molecule, antibody, aptamers, siRNA or antisense oligonucleotide.

The PARP inhibitor is selected from the group consisting of: iniparib (BSI 201), talazoparib (also known as BMN-673), velipari (ABT-888), olaparib (also known as AZD-2281 and commercialized as Lynparza®), rucaparib (also known as Rubraca®) or niraparib (also known as Zejula®).

The PI3K, MAPK, PAK, mTOR, TKI, PARP and/or EGFR inhibitors as described above can be used as part of a multi-therapy for the treatment of proliferative glomerulonephritis in a subject in need thereof.

The PI3K inhibitor can be used alone as a single inhibitor or in combination with other inhibitors like MAPK, PAK, mTOR, TKI, PARP and/or EGFR inhibitors. When several inhibitors are used, a mixture of inhibitors is obtained. In the case of multi-therapy (for example, bi-, tri- or quadritherapy), at least one other inhibitor can accompany the PI3K inhibitor.

In a particular embodiment, the PI3K and MAPK inhibitors can be combined as a bi-therapy for use in the treatment of proliferative glomerulonephritis. In a particular embodiment, the PI3K and MAPK inhibitors can be combined for use as a bi-therapy, wherein the PI3K and MAPK inhibitors are BYL719 and selumetinib respectfully.

In another embodiment, the PI3K and ERK inhibitors can be combined as a bi-therapy for use in the treatment proliferative glomerulonephritis. In a particular embodiment, the PI3K and ERK inhibitors can be combined for use as a bi-therapy, wherein the PI3K and ERK inhibitors are BYL719 and VTX-11e respectfully.

In another embodiment, the PI3K and mTOR inhibitors can be combined as a bi-therapy for use in the treatment proliferative glomerulonephritis. In a particular embodiment, the PI3K and mTOR inhibitors can be combined for use as a bi-therapy, wherein the PI3K and mTOR inhibitors are BYL719 and everolimus respectfully.

In another embodiment, the PI3K and TK inhibitors can be combined as a bi-therapy for use in the treatment proliferative glomerulonephritis. In a particular embodiment, the PI3K and TK inhibitors can be combined for use as a bi-therapy, wherein the PI3K and TK inhibitors are BYL719 and sunitinib respectfully.

In another embodiment, the PI3K and VEGF inhibitors can be combined as a as a bi-therapy for use in the treatment proliferative glomerulonephritis. In a particular embodiment, the PI3K and TK inhibitors can be combined for use as a bi-therapy, wherein the PI3K and VEGF inhibitors are BYL719 and brolucizumab (RTH258) respectfully.

In another embodiment, the PI3K, MAPK and PAK inhibitors can be combined as a tri-therapy for use in the treatment of proliferative glomerulonephritis. In a particular embodiment, the PI3K, MAPK and PAK inhibitors can be combined as a tri-therapy, wherein the PI3K, MAPK and inhibitors are BYL719, selumetinib and IPA-3 respectfully.

As used herein the terms “administering” or “administration” refer to the act of injecting or otherwise physically delivering a substance as it exists outside the body (e.g., an inhibitor of PI3K) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

As used herein, the term “administration simultaneously” refers to administration of at least 2 or 3 active ingredients by the same route and at the same time or at substantially the same time. The term “administration separately” refers to an administration of at least 2 or 3 active ingredients at the same time or at substantially the same time by different routes. The term “administration sequentially” refers to an administration of at least 2 or 3 active ingredients at different times, the administration route being identical or different.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The PIK3CA inhibitor alone or combined with a classical treatment, as described above may be combined with pharmaceutically acceptable excipients, and optionally sustained-release matrices, such as biodegradable polymers, to form pharmaceutical compositions.

Accordingly, the invention relates to a pharmaceutical composition comprising a PIK3CA inhibitor for use in the treatment of proliferative glomerulonephritis as described above.

In a particular embodiment, the invention relates to a pharmaceutical composition comprising a PIK3CA inhibitor for use in the treatment of lupus nephritis (LN).

In a particular embodiment, the invention relates to a pharmaceutical composition comprising a PIK3CA inhibitor for use in the treatment of Focal and Segmental Glomerulosclerosis (FSGS).

In a further embodiment, the invention relates to a pharmaceutical composition comprising i) a PIK3CA inhibitor and ii) a classical treatment as described above as combined preparation to treat proliferative glomerulonephritis.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

A further object of the present invention relates to a method of screening a drug suitable for the treatment of proliferative glomerulonephritis comprising i) providing a test compound and ii) determining the ability of said test compound to inhibit the activity of PI3K.

Any biological assay well known in the art could be suitable for determining the ability of the test compound to inhibit the activity of PI3K. In some embodiments, the assay first comprises determining the ability of the test compound to bind to PI3K. In some embodiments, a population of cells is then contacted and activated so as to determine the ability of the test compound to inhibit the activity of PI3K. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity of PI3K, as determined using in vitro methods described herein, are likely to exhibit similar modulatory capacity in applications in vivo. Typically, the test compound is selected from the group consisting of peptides, peptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1: PIK3CA gain of function mutation leads to progressive glomerular disease. Quantification of glomerular score index, and Ki67. AU: Arbitrary unit.

FIG. 2: Alpelisib improves kidney lesions in uninephrectomized PIK3CAPod-HO mice. A. Urinary albumin to creatinine ratio of PIK3CAWT and PIK3CAPod-HO mice 2 weeks following uninephrectomy and treated either with vehicle or alpelisib (n=8-9 mice per group). B. Serum creatinine levels, C. blood urea nitrogen (BUN) levels of PIK3CAWT and PIK3CAPod-HO mice 2 weeks following uninephrectomy and treated either with vehicle or alpelisib (n=8-9 mice per group). D. Glomerular lesion score of PIK3CAWT and PIK3CAPod-HO mice 2 weeks following uninephrectomy and treated either with vehicle or alpelisib (n=8-9 mice per group). E. Trajectory of albumin to creatinine ratio in the urine of PIK3CAPod-HO mice following uninephrectomy and treated with either vehicle or alpelisib (n=8-9 mice per group).

FIG. 3: Alpelisib improves kidney lesions in an accelerated mouse model of collapsing glomerulopathy. Scale bar P-S6RP, and P-AKTSer473 mean intensity quantification (n=5-6 mice per group). P. Serum creatinine level, and Blood urea nitrogen level of Tg26WT and Tg26HE mice at the time of sacrifice (4 weeks following uninephrectomy and treated either with vehicle or alpelisib) (n=8-9 mice per group)

FIG. 4: Alpelisib improves kidney lesions in Lupus Nephritis models. A. Urinary albumin to creatinine ratio B. Blood urea nitrogen level, C. kidney to body weight ratio of NZBWF1/J mice at the time of sacrifice (4 weeks following either sham operation or uninephrectomy followed by the treatment either with vehicle or alpelisib) (n=5-15 mice per group). D. Representative P-S6RP/Nephrin coimmunofluorescence staining of kidneys from 4 weeks following either sham operation or uninephrectomy followed by the treatment either with vehicle or alpelisib) (n=4-6 mice per group). Scale bar 20 mm. E. P-S6RP mean intensity quantification (n=4-6 mice per group). F. Col3a, G. Col1a, and H. Tnfα quantification of qRT-PCR analysis in the kidney cortex of kidneys at the time of sacrifice (4 weeks following either sham operation or uninephrectomy followed by the treatment either with vehicle or alpelisib) (n=5-15 mice per group). I. Kaplan-Meier survival curves of MRL-lpr mice treated either with vehicle or alpelisib (n=13-14 mice per group). J. Urinary albumin to creatinine ratio of MRL-lpr mice treated either with vehicle or alpelisib from 8 weeks old till death (n=13-14 mice per group). K. Vehicle treated mice, and L. alpelisib treated mice of the trajectory of urinary albumin to creatinine ratio of MRL-lpr mice (n=13-14 mice per group).

FIG. 5: PIK3CA inhibitors improve kidney lesions in a Lupus Nephritis model. (A) Glomerular injury score (max severity is 4), (B) blood urea (Blood Urea Nitrogen, norm <8-10 mol/l) and (C) albuminuria/creatinuria ratio (mg/mmol) in mice nephrectomised at 24 weeks of age and treated for 4 weeks (daily gavage). ANOVA test was used to determine the statistical significance between experimental groups.

### EXAMPLE 1

### Material & Methods

### Animal Studies

R26StopFLP110* (Stock #012343), R26StopCAG-EGFP (Stock #006071), and Podocin-Cre mice (Stock #008523) on the C57BL/6 background, Tg26/HIV mice (Stock # 022354) on FVB background, Pik3Calox/lox mice (Stock #017704), MRL/MpJ-Faslpr/J (Stock #000485), were obtained from the Jackson Library. NZBWF1/OlaHsd mice were obtained from Envigo. Whenever required, at least 10 backcrossings were done before using mice for experiments. Mice were randomly allocated to each group in sex-, age-, and body weight-matched manner, except otherwise indicated. All animal procedures were approved by the Ministère de l'Enseignement Supérieur, de la Recherche et de l'Innovation (APAFIS #30133-2020111914293579 v8) and performed in accordance with the guidelines of Paris Descartes University to ensure the animal welfare.

For the uninephrectomy experiments, the right kidneys were removed under anesthesia. For the medical treatment of mice, 50 mg kg −1 alpelisib (MedChem Tronica) in 1% carboxymethylcellulose (Sigma Aldrich)+0.5% Tween (Sigma Aldrich) or vehicle (1% carboxymethylcellulose+0.5% Tween) was administered by oral gavage daily for the indicated periods of time. Blood and urines were obtained at the indicated times. At euthanasia, blood, urines, kidneys were harvested. In some experiments, spleens, hearts, and bone marrow (BM) in femurs and tibias were harvested as well. Tissues were fixed in 4% paraformaldehyde and paraffin embedded for immunohistochemical analysis, snap-frozen in Optimal Cutting Temperature (OCT), or stored at −80° C. for mRNA or protein analysis.

### Blood and Urine Measurement

Mouse blood counts were analyzed using a hematology analyzer (ProCyte Dx; IDEXX Laboratories). Mouse serum creatinine, blood urea nitrogen, urinary albumin, and urinary creatinine were evaluated using an AU5800 (Beckman Coulter) autoanalyzer. Serum anti-dsDNA measurement was performed according to the manufacturer's instruction using mouse anti-dsDNA ELISA kit (LBIS). The absorbance was measured using Ininite M Nano (TECAN).

### Histopathology and Immunohistochemistry Analysis

4-μm kidney sections were stained with periodic acid-Schiff (PAS), Masson's Trichrome (MT), Periodic acid metenamine silver (PAM) or Hematoxylin and Eosin (HE) staining for histological analysis. The degree of glomerular lesions was evaluated using the following scoring system: 0=no lesion, 1=affecting up to 25% of the glomerulus, 2=affecting 25-50% of the glomerulus, 3=affecting 50-75% of the glomerulus, and 4=affecting 75-100%. At least 50 glomeruli per sample were measured for each analysis. An indirect immunoperoxidase method was used for immunohistochemistry. Briefly, after deparaffinization, antigen retrieval was performed with citrate buffer (pH6) or Tris-EDTA buffer (pH9 or pH6) using microwave or high temperature (95° C.). Endogenous peroxidase activity was quenched using 3% hydrogen peroxide; nonspecific protein binding was blocked using 2.5% normal horse serum (Vector Laboratories), and endogenous biotin activity was quenched using the Avidin/Biotin Blocking Kit (Vector Laboratories). When using mouse primary antibodies on mouse tissue, Klear mouse blocking reagent (Diagomics) was used to block endogenous mouse immunoglobulins. After blocking, the tissue sections were incubated with the primary antibodies overnight at 4° C. The following primary antibodies were used: rabbit anti-Ki67 antibody (SP6; Thermo Fisher Scientific), rabbit anti-P-S6RP antibody (D68F8; Cell Signaling Technology), mouse anti-P-AKT (Ser473) antibody (587F11; Cell Signaling Technology), rabbit anti-P-AKT (Thr308) antibody (C31E5E; Cell Signaling Technology), mouse anti-S6RP antibody (54E2; Cell Signaling Technology), chicken anti-GFP antibody (Abcam), guinea pig anti-Nephrin antibody (GP-N2; Progen), rabbit anti-Podocin antibody (P0372; Sigma Aldrich), mouse anti-WTI antibody (6F-H2; DAKO). The corresponding secondary antibodies including anti-mouse IgG, anti-rabbit IgG, anti-guinea pig IgG, biotinylated anti-rabbit IgG (Vector Laboratories), and biotinylated anti-mouse IgG (Vector Laboratories) were applied. For Avidin/Biotin detection, an R.T.U. Vectastain Kit (Vector Laboratories) or streptavidin (Thermo Fisher Scientific) was used. The immunoreactive antigens sites were detected with hydrogen peroxide and diaminobenzidine. For frozen immunofluorescent staining, frozen tissue sections (4 μm) were briefly air dried and fixed in 50% methanol/50% acetone. The sections were then blocked with 2.5% normal horse serum and incubated with primary antibodies: guinea pig anti-Nephrin antibody (GP-N2; Progen) overnight at 4° C. The sections were then incubated at room temperature for 40 min with secondary antibodies: fluorescein isothiocyanate-conjugated (FITC) anti-mouse IgG (Sigma Aldrich), FITC anti-mouse IgM (BD Biosciences), or FITC anti-mouse C3 antibody (abcam). The images were captured on a Zeiss LSM700 confocal microscope. Immunohistochemistry revelation was performed with appropriate horseradish peroxidase (HRP) antibodies and images were captured with a Nikon Eclipse E800 microscope. Image J software was used for image analysis. Ilastik (interactive machine learning for (bio) image analysis, version 1.33post3) was used to select the positive signal area in the glomeruli.

cDNA Synthesis and Quantitative RT-PCR Analysis

Total RNA in kidney cortex was extracted using NucleoSpin RNA (Macherey Nagel). Complementary DNA was reverse-transcribed using TaqMan high-capacity cDNA RT kit (Thermofisher). qPCR was performed with iTaq universal SYBR Green Supermix (Bio-Rad Laboratories) using CFX Connect real-time system (Bio-Rad Laboratories). Expression levels were analyzed by delta-delta Ct method. Hypoxanthine phosphoribosyltransferase (Hprt) was used as the normalization control.

### Western Blotting

Protein extracts in RIPA buffer from the kidney cortex were separated by SDS-PAGE, transferred onto the membrane and incubated with antibodies, and followed by appropriate peroxidase-conjugated secondary antibody incubation. Following primary antibodies were used: rabbit anti-P-S6RP antibody (D68F8; Cell Signaling Technology), rabbit anti-P-AKT (Ser473) antibody (D9E; Cell Signaling Technology), rabbit anti-P-AKT (Thr308) antibody (C31E5E; Cell Signaling Technology), mouse anti-S6RP antibody (54E2; Cell Signaling Technology), mouse anti-Akt (pan) antibody (40D4, Cell Signaling Technology), chicken anti-GFP antibody (ab13970; Abcam), mouse anti-alpha-tublin antidbody (B-5-1-2; Sigma Aldrich), rabbit anti-Nephrin antibody (29070; IBL), rabbit anti-Podocin antibody (P0372; Sigma Aldrich). Chemiluminescence was acquired using ChemiDoc MP (Bio-Rad Laboratories) and densitometry was performed using the Image Lab software (Bio-Rad Laboratories, version 6.0.1).

### Mouse Cell Preparation and Flow Cytometry

Mononuclear cells (MNCs) from peripheral blood, spleen, BM, and lymph nodes of MRL/Lpr mice treated either with vehicle or alpelisib were prepared essentially as described before 12. For lymphocyte and granulocyte/macrophage lineage analysis, anti-CD3a antibody (500A2; Becton Dickinson), anti-CD4 antibody (L3T4; Becton Dickinson), anti-CD8a antibody (63-6.72; Becton Dickinson), anti-B220 antibody (RA3-6B2; Becton Dickinson), anti-CD11b antibody (M1/70; Becton Dickinson), anti-Ly6G antibody (RB6-8C5; Becton Dickinson), and CD16/CD32 (2.4G2; Becton Dickinson). Cells were resuspended in 0.2 to 1 mL of PBS+2% FCS+2 uL of 7-aminoactinomycine D (7-AAD, Thermo Fischer Scientific). Flow cytometry was performed using SP6800 Spectral Analyzer (SONY). Data were analyzed with FlowJo software (TreeStar).

### Preparation of Single Cell Suspension

For Imaging flow cytometry (Amnis ImageStream) or single-cell RNAseq analysis, single cell suspensions were prepared as follows. PIK3CAWT and PIK3CAPodo-HT mice were intravenously injected with M-450 dynabeads (Thermofischer, ref #14013) and saline. Kidneys were rinsed in phosphate-buffered saline and cut into small pieces in RPMI1640 media on ice. Multi Tissue Dissociation Kit 1 (Miltenyi Biotec) was used for digesting the kidney. First, in order to enrich the glomeruli, kidney pieces were stirred in the digestion buffer for 20 min at 37 degrees. Cells were then filtered (100 μm, Miltenyi Biotec) and centrifuged 5 min at 300 g at 4 degrees. Cell pellets were resuspended in RPMI 1640 media and rinsed. The cell pellets were again resuspended in the digestion buffer, and digested with the half protocol 37C_Multi_B to make the single cell suspension. Kidney cell suspensions were filtered (70 um, Miltenyi Biotec), centrifuged 5 min at 300 g at 4 degrees, and resuspended in RPMI 1640 media. Cell pellets were incubated with RBC lysis buffer (Miltenyi Biotec) on ice for 3 min, centrifuged at 300 g for 5 min at 4 degrees, and resuspended in 1 ml PBS+0.04% BSA. Cell number and viability were analyzed under microscope with 0.4% Trypan Blue solution. The protocol was elaborated to minimize the experimental time to achieve the single cell suspension with the maximum possible cell viability.

### Imaging Flow Cytometry (Amnis ImageStream)

Following single cell suspension procedure, flow cytometry cell preparation was done essentially following the Flow Cytometry Protocol by Cell Signaling Technology. Briefly, cell samples were fixed with 4% PFA for 15 min, permeabilized with methanol on ice, and resuspended in PBS after several washes. Samples were labelled with rabbit anti-P-S6RP-AF647 antibody (D68F8; Cell Signaling Technology) and/or rabbit anti-P-AktThr308-PE antibody (D25E6; Cell Signaling Technology). Rabbit IgG isotype controls (AF647 and PE; Cell Signaling Technology) were used. Resuspended cells were run on an ImageStream ISX mkII (Amnis) that combines flow cytometry with detailed cell imaging. Magnification (40×) was used for all acquisitions. Data were acquired with INSPIRE software (Amnis) and analyzed with IDEAS software (v.6.2, Amnis).

Single Cell RNA-Seq Barcoding and cDNA Synthesis

The single cell suspension, with approximately 20,000 cells, were used as input on the 10× Chromium Controller to target to obtain 10,000 cells per library (10× Genomics). Barcoding and cDNA synthesis were done following the manufacturer's instructions (10× Genomics). An equimolar pool of the 4 individual 10× Genomics Single Cell Expression 3′ V3 libraries was prepared by the Imagine Genomic Core Facility and sequenced on an S2 FlowCell using the NovaSeq6000, Illumina (sequencing mode Paired-End 100+100 bases+indexes). A total of 2 Billions reads was targeted for this pool of 4 libraries (500 Millions reads per library).

### Single Cell RNA-Seq Pre-Processing

Sequencing data quality analysis was performed using FastQC v0.11.2 on 4 mouse samples. For read alignment and unique molecular identifiers (UMI) quantification, CellRanger software v3.0.2 was used on Mus Musculus genome mm10 with default parameters and gene annotation from Ensembl 100 (plus eGFP and tdTomato). The 4 expression matrices containing the UMI counts were merged, and only the genes with UMI≥1 in at least one cell were kept. The following filters were applied to generate a global matrix used in further analysis: cells with UMI≥2000, number of detected genes≥600, and cells with UMI in mitochondrial genes≤60%. For UMIs normalization, Seurat 3.1.1 was used (Butler, A., Hoffman, P., Smibert, P., Papalexi, E. & Satija, R. Integrating single-cell transcriptomic data across different conditions, technologies, and species. Nature biotechnology 36, 411-420 (2018)), and global-scaling normalization method was applied with a scale factor of 10,000 and log-transformation of data. This was followed by a scaling linear transformation step, to avoid highly-expressed genes having higher weight in downstream analysis.

### Clustering and Marker Genes

PCA was performed on the scaled data, with a Jackstraw plot to choose how many PCs to retain as an input for Seurat clustering step. Clustering step was performed using default parameters, Louvain algorithm as the clustering method, 15 PC and a resolution parameter defining the clusters granularity set to 0.5. Marker genes defining each cluster were found via differential expression testing, with a Wilcoxon rank sum test and a log fold change threshold of 1.

### Trajectory Analysis

Monocle single-cell trajectory was constructed using M3Drop (Andrews, T. S. & Hemberg, M. M3Drop: dropout-based feature selection for scRNASeq. Bioinformatics 35, 2865-2867 (2019)) and Monocle 2.10.1 (Qiu, X. et al. Reversed graph embedding resolves complex single-cell trajectories. Nature methods 14, 979-982 (2017)). Input genes for Monocle trajectory construction were selected using an unsupervised approach via M3Drop result, which identifies differentially expressed genes based on a Michaelis-Menten function for the relationship between mean expression and dropout rate, the relevant genes being the ones shifting above a fitted curve. The default Monocle workflow was then performed to generate the trajectories.

### Enrichment Analysis

Analysis for enriched KEGG pathways were performed using WebGestaltR package (Qiu, X. et al. Reversed graph embedding resolves complex single-cell trajectories. Nature methods 14, 979-982 (2017)), on databases from Mus Musculus organism. GO terms and pathways were considered as enriched if fold enrichment≥2.0, uncorrected p-value≤0.05 and minimum number of regulated genes in pathway/term≥2.0.

### LC-MS Analysis of Alpelisib

Each tissue type was homogenized in 100% cold methanol with tissue-to-solvent ratio of 1-mg tissue to 5-μl methanol. After sonication of 20 s, the tissue extract was centrifuged at 13,000 g for 30 min and then injected onto a Phenomenex Kinetex XB-C18 HPLC column (100 mm by 2.1 mm) at 45° C. Alpelisib was analyzed by reverse-phase HPLC (Shimadzu LCMS system 8040 interfaced with the LabSolutions software). Twenty micrograms of tissue extracts were injected onto a column, and the mobile phase used for the separation consisted of two eluants: Solvent A was 0.1% formic acid in ddH20, and solvent B was acetonitrile with 0.1% of formic acid. Compounds were separated by the following discontinuous gradient at a flow rate of 0.6 ml/min: The initial concentration of 20% in solvent B increase to 70% over 6 min, and this was followed by a decrease to 20% over the next minute, and the initial conditions were then maintained for 14 min. The alpelisib was monitored spectrophotometrically by absorbance (photodiode detector) from 205 to 600 nm and by tandem mass detection. The mass measurement was implemented in positive ion mode using multiple reaction monitoring (MRM) with an electrospray ionization source. Three MRM transitions for alpelisib are used as follows: 442.1>328.0, 442.1>288.0, and 442.1>115.1. The quantification was done by integration of the peak absorbance area using a calibration curve established with various known concentrations of alpelisib.

### Human Samples

Human kidney samples were obtained from patients followed at Hôpital Necker Enfants Malades. Clinical and biological data available at the time of the kidney biopsy, or lupus nephritis flare visit were collected. The classical histological and immunofluorescence analysis were performed with kidney biopsies fixed with formalin, alcohol, and acetic acid (AFA), which were subsequently paraffin-embedded. Some of the immunofluorescence studies and droplet digital PCR (ddPCR) were performed with OCT-frozen sections. Written informed consent was obtained from each patient.

### STAR-FISH and Droplet Digital PCR

STAR-FISH (Specific-To-Allele PCR-FISH) was performed as previously described¹³. For droplet digital PCR (ddPCR), 20 μm frozen kidney sections were mounted on PEN-membrane slides and rapidly stained with hematoxylin to recognize the kidney structure. Laser capture microdissection (LCM) was performed using a Leica LMD7000 system. Glomeruli or tubules were collected for each patient. ddPCR (QX200 system, Bio-Rad Laboratories) was performed to detect specifically the variant in the different pools of cells. The ddPCR Supermix for probes (no dUTPs) were used according the manufacturer's protocol. Data were analyzed using the QX200 droplet reader and Quantasoft Analysis Pro software (BioRad Laboratories; version 1.0.596).

### Spatial Transcriptomics

GeoMx™ digital spatial profiling experiments were performed according to the Nanostring GeoMx-NGS DSP instrument manual and as previously reported (Merritt, C. R. et al. Multiplex digital spatial profiling of proteins and RNA in fixed tissue. Nature biotechnology 38, 586-599 (2020)). Briefly, 4 um AFA-fixed paraffin-embedded human samples were baked overnight at 37° C. and 1 hour at 65° C., and then they have been processed on Leica automation platform with a protocol included three major steps: 1) slide baking, 2) Antigen Retrieval 20 min at 100° C., 3) 1.0 ug/ml Proteinase K treatment for 15 min. After taking the slide off Leica, slides were incubated with GeoMx WTA assay probe cocktail overnight. The following day, slides were washed and applied with morphology marker incubation before loading onto GeoMx machine. Anti-pan-cytokeratin (clone AE1/AE3, Novus Biologicals), anti-CD10 (clone EPR22867-118, Abcam), anti-CD31 (clone JC/70A, abcam) were used as morphology markers. On the GeoMx machine, slides were fluorescently scanned and finished ROI selection. Barcodes were collected and subsequent barcoding reading were performed on Illumina NGS platform. Individual counts were normalized against the 75th percentile of the signal from their own ROI (Q3 Normalization). Data analysis was performed on GeoMx DSP software v2.5.1.145. Reactome database v78 was used for pathway analysis.

### Data Analysis and Statistics

Data were expressed as the means±SEM or SD. Survival curves were analyzed with the Log-rank (Mantel-Cox) test. Two-way ANOVA with Bonferroni's post hoc test was used to determine the statistical significance between experimental groups. A two-tailed Student's t test was used to compare two-group experiments. The statistical analysis was performed using GraphPad Prism software (version 7.0a).

### Results

### A Patient With PIK3CA Gain of Function Mutation in Podocytes

The kidney biopsy of patient 1 revealed the presence of a complex glomerulonephritis characterized by focal and segmental glomerulosclerosis (FSGS), mixing collapsing lesions with crescentic formation without immune deposits, extensive fibrosis (>80% of the parenchyma) with tubular dilation, casts and inflammatory infiltrating cells (data not shown). Immunofluorescence studies showed proliferation and activation of the AKT/mTOR pathway in glomerular epithelial cells (data not shown). We initially thought that these lesions were resulting from the complex medical situation combining vesicoureteral reflux, congestive heart failure and rapamycin. We stopped rapamycin and did not observe any proteinuria or kidney function improvement (data not shown). As previously reported, because of the severity of the CLOVES/PROS condition, we were authorized to treat this patient with alpelisib, an approved specific PIK3CA inhibitor. The drug led to a shrinkage of the different malformations and full recovery of the congestive heart failure. Intriguingly, we observed improvement of the high amount of proteinuria and the stabilization of the kidney function. We hypothesized that this patient could carry PIK3CA mutation in kidney epithelial cells. To explore this hypothesis, we first started by performing in situ PCR using fluorescently labelled mismatched primers designed to specifically amplify mutant (H1047R) and wild type PIK3CA alleles on paraffin embedded kidney biopsy section. T-47D cells were used as positive controls (data not shown). We observed the presence of PIK3CA mutant alleles in glomerular epithelial cells (data not shown). Several types of glomerular cells seemed to carry the mutation including podocytes. However, we were not able to perform any coimmunostaining to highlight the type of cells that were affected. To confirm the presence of the mutation, we performed droplet PCR of glomerulus isolated from the kidney biopsy from patient 1 using laser capture microdissection (data not shown). Whereas control biopsies did not reveal any alteration in PIK3CA alleles, droplet PCR of the glomeruli from patient 1 demonstrated the presence of PIK3CA H1047R mutation (data not shown). We concluded that patient 1 was carrying PIK3CA gain of function mutation in glomerular epithelial cells.

### A Mouse Model of PIK3CA Gain of Function Mutation in Podocytes

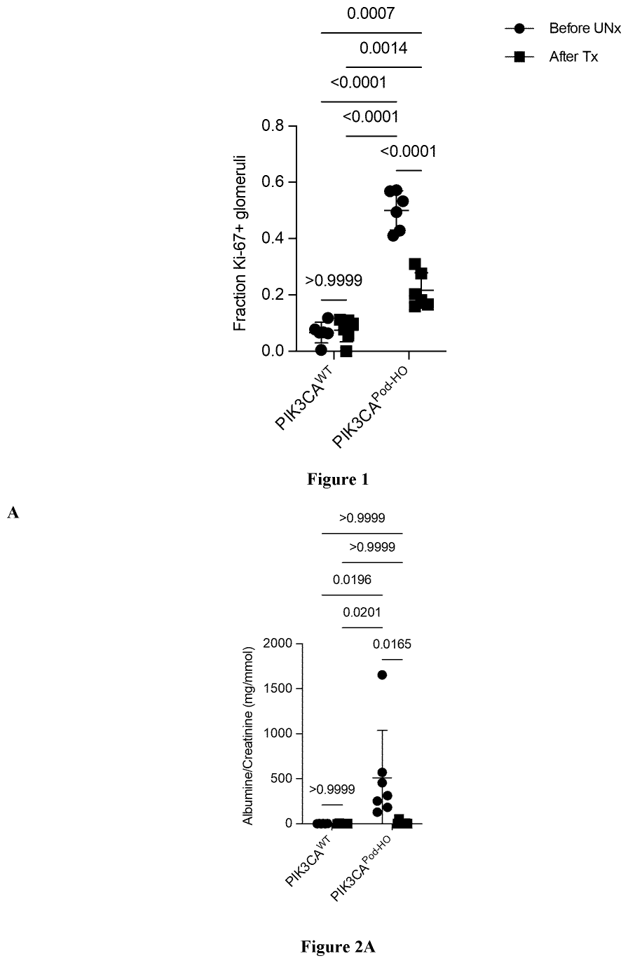

Based on the glomerular profile of the kidney dysfunction of patient 1, the presence of the PIK3CA variant in glomerular cells and the drastic reduction of proteinuria following alpelisib introduction, we decided to create a mouse model of PIK3CA gain of function mutation specifically in podocytes. We took advantage of the transgenic mouse strain, R26StopFLP110*, which, after breading with Cre recombinase mice, express a dominant active PIK3CA transgene. R26StopFLP110* mice were crossed with Podocin Cre mice to generate PIK3CA^{Pod} animals that express the over-activated form of PIK3CA. In order to follow the Cre recombination, PIK3CA^{Pod-HET} mice were then interbred with Gt(ROSA)26Sor^{tm4(ACTB-tdTomato,-EGFP)Luo/J} mice¹¹. These mice express in all tissues a cell membrane localized tdTomato fluorescent protein, that is replaced by GFP after Cre recombination. At birth, PIK3CA^{Pod-HET} mice were undistinguishable from WT control littermates (PIK3CA ^{WT}). However, PIK3CA^{Pod-HET} mice progressively developed albuminuria from 3 months of age with slowly declining kidney function (data not shown) and reduced survival (data not shown). Compared to controls, PIK3CA^{Pod-HET} mice demonstrated glomerular lesion mixing crescentic formation and collapsing glomerulopathy such as observed in patient 1 (data not shown). Optical microscopy revealed hypertrophic multinucleated podocytes with intracytoplasmic vacuoles, but also protein casts and inflammatory cell infiltrations (data not shown). Using immunofluorescence experiments, we confirmed that, the AKT/mTOR pathway was activated in podocytes of PIK3CA^{Pod-HET} mice (data not shown). One of the main characteristics of podocyte injury is the loss of differentiation markers. We decided to study cellular differentiation by analyzing the expression of WT1, a transcription factor that marks mature podocytes, nephrin, podocin and nestin, proteins contributing to foot process formation in differentiated podocytes. Staining experiments showed a profound loss of podocytes differentiation markers in PIK3CA^{Pod-HET} mice compared to controls (data not shown). Mechanistically, PIK3CA is involved in cell growth and proliferation. We first investigated the proliferation rate in the glomeruli of the different mouse models using Ki-67, a proliferation marker. Whereas the number of Ki-67+ cells was low in control mice, the number of Ki-67+ cells was markedly increased in glomeruli of PIK3CA^{Pod-HET} mice (data not shown). Then, using Amnis ImageStream® system, we confirmed that podocytes isolated from PIK3CAP^{Pod-HET} mice were hypertrophic compared to controls (FIG. 1M). Flow cytometry experiments showed a correlation between podocyte size and the degree of activation of the AKT/mTOR pathway, notably on mTORC1 pathway (data not shown). These data are consistent with the fact that cells entering the cell cycle lose their differentiation markers. Indeed, we concluded that overactivation of PIK3CA in podocytes leads to both proliferation and dedifferentiation, which results in FSGS with progressive proteinuria and kidney dysfunction. This mouse model recapitulates the kidney phenotype observed in patient 1.

In order to explore the allele dosage effect of overactivation of PIK3CA pathway, we generated homozygous mutant for PIK3CA in podocytes (referred here after to PIK3CA^{Pod-HO} mice). At birth PIK3CA^{Pod-HO} mice were indistinguishable from controls. However, these mice rapidly developed a high amount of albuminuria (data not shown) with reduced survival (data not shown). Histological examination at the age of 12 weeks revealed severe proliferative glomerulonephritis (data not shown) along with podocyte dedifferentiation (data not shown). Consistently, dedifferentiation, AKT/mTOR pathway recruitment and proliferation were observed in podocytes (data not shown). These findings demonstrate that the cumulative activation of the PIK3CA pathway is associated with a more severe disease phenotype.

### Overactivation of PIK3CA Pathway in Podocytes is Associated With Changes in Cell Fate Determination

Podocytes are post mitotic cells with limited possibilities of proliferation and renewal. However, under certain pathological circumstances, podocytes can reinter into the cell cycle and become dedifferentiated (Griffin, S. V., Petermann, A. T., Durvasula, R. V. & Shankland, S. J. Podocyte proliferation and differentiation in glomerular disease: role of cell-cycle regulatory proteins. Nephrol Dial Transplant 18 Suppl 6, vi8-13 (2003)). To identify the early transcriptional changes in podocytes associated with podocyte PIK3CA gain-of-function mutation, we performed single-cell RNA sequencing (scRNA-seq) and mapped kidney cells from PIK3CA^{WT} and PIK3CA^{Pod-HET} mice (data not shown). In order to increase the podocyte population, which counts only 0.18% of normal mouse kidney (Park, J. et al. Single-cell transcriptomics of the mouse kidney reveals potential cellular targets of kidney disease. Science (New York, N.Y 360, 758-763 (2018)), we enriched the glomeruli with magnetic beads injection. Two mice for each condition were pooled in order to cover the heterogeneity of phenotype. Total of 49,600 cells were isolated and sequenced. After data normalization and filtering (data not shown), 26,508 cells were further analyzed. Unsupervised clustering of the entire pooled dataset identified 20 clusters (data not shown). With well-established cell-type markers from the literature, we annotated broad cluster classes (data not shown). We annotated 6 podocyte clusters based on the expressions of Nphs2, Synpo, Podxl, Pdpn, and EGFP (data not shown). We decided to include some of the clusters with weaker expressions of podocyte markers (Podocyte-4,-5, and -6), with aim to understand different cell states of podocytes. Monocle single-cell trajectory from 6 podocyte clusters resulted in 5 distinct clusters (data not shown). Whereas podocyte-2, 4, and 5 cluster showed similar pseudotemporal ordering distributions, podocyte-1 and 3 clusters were distinct (data not shown). Podocyte-1 cluster was activated for PI3K-AKT pathway as well as signaling pathways regulating pluripotency; Podocyte-3 cluster was enriched for inflammatory pathways (data not shown). While in PIK3CA^{WT} mice podocyte clusters accounted for about 10% of the whole analyzed cells, in PIK3CAP^{Pod-HET} mice, it increased to 23.7%, demonstrating the proliferation of podocytes (data not shown). We finally observed that podocytes of PIK3CA^{Pod-HET} mice showed increased expression of genes that are known to be upregulated in FSGS such as metallothioneins Mt1 and Mt2, EGF, wnt4, Col1a2, Col4a3, Col4a4, or Il18, who are related to inhibition of apoptosis, dedifferentiation, and proliferation (data not shown). These results indicate that PI3K-AKT pathway activation in podocytes drives podocyte fate transition.

### Alpelisib Improves PIK3CA^{Pod} Mouse Models

Patient 1 demonstrated albuminuria improvement following alpelisib introduction, but, in the meantime, alpelisib was associated with vascular malformations shrinkage and correction of the severe congestive heart failure adding potential confusing factors. We wondered if alpelisib could be a therapeutic option. We first explored if the drug was able to diffuse into kidneys. Liquid chromatography-mass spectrometry (LC-MS) analysis revealed that alpelisib was detectable 2 hours after oral gavage (data not shown). We next administered alpelisib orally during 2-3 weeks to 3 months old PIK3CA^{Pod-HET} mice and observed a progressive reduction in albuminuria (data not shown) with glomerular inhibition of the AKT/mTOR pathway (data not shown) and podocyte size reduction (data not shown).

scRNA-seq analysis of kidney cells from alpelisib-treated PIK3CA^{Pod-HET} mice revealed that the podocyte populations were restored to 11.7% compared to vehicle-treated PIK3CA^{Pod-HET} mice (data not shown). In addition, we observed that alpelisib treatment was associated with correction of various gene expression anomalies (data not shown). These results indicate that alpelisib was able to successfully interfere with podocyte fate transition induced by the overactivation of the PI3K-AKT pathway.

Then, we started a pilot study by administering either, vehicle or alpelisib to PIK3CA^{Pod-HO} mice at the age of 3 months when the mice have already established proteinuria. We decided to use PIK3CA^{Pod-HO} mice, since disease more severe that in the heterozygous model. Three months old PIK3CA^{Pod-HO} mice were treated during 2 consecutive weeks (data not shown). During the treatment period, we observed a progressive albuminuria reduction in the alpelisib treated mice. Mice were then sacrificed and kidney histological analysis showed that PIK3CA^{Pod-HO} mice treated with alpelisib had preserved glomeruli compared to PIK3CA^{Pod-HO} mice treated with vehicle (data not shown). Podocyte differentiation markers were still expressed in alpelisib treated group at protein levels (data not shown). AKT/mTOR pathway was blunted in podocytes of PIK3CA^{Pod-HO} mice (data not shown). Mechanistically, alpelisib treatment was associated with a decrease in glomerulus proliferation (FIG. 1 and data not shown).

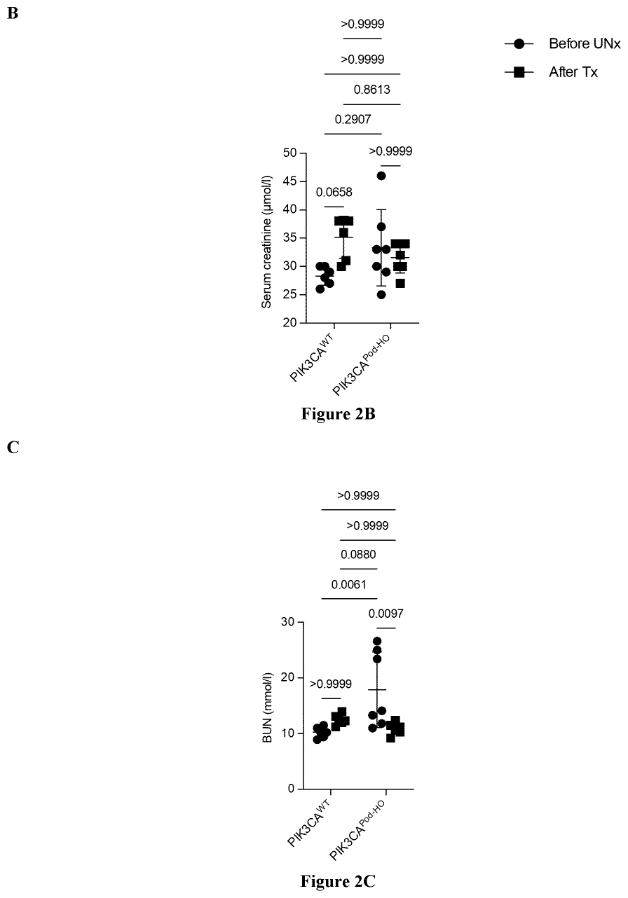

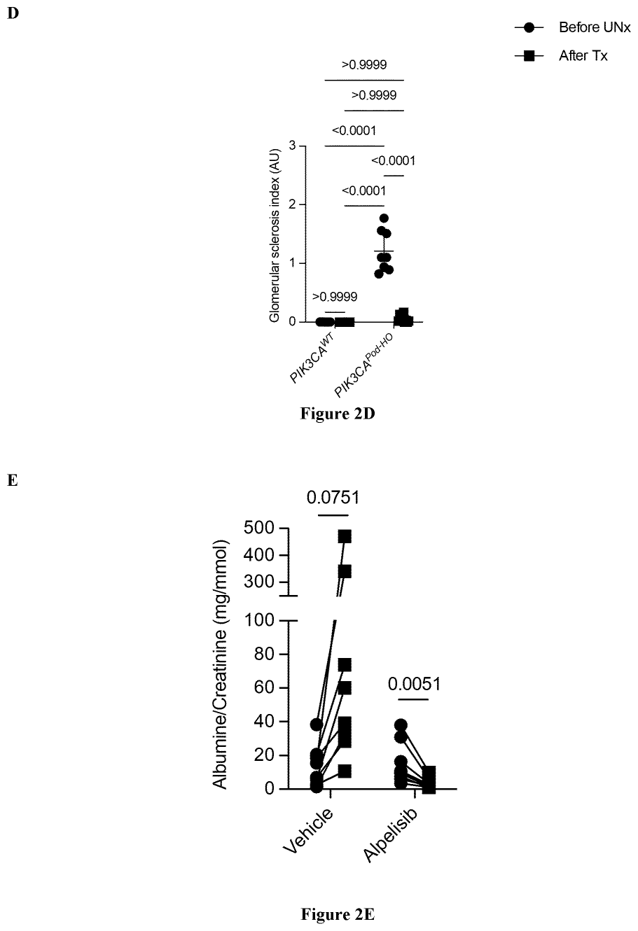

In order to better compare the improvement and potential reversibility of kidney lesions, we decided to perform unilateral nephrectomy in PIK3CA^{Pod-HO} mice and controls at the age of 8 weeks old, to treat them with either vehicle or alpelisib, and to paired compare the kidney at the time of sacrifice (data not shown). Following nephrectomy or sham surgery, mice were randomly treated with either vehicle or alpelisib during 2 weeks (data not shown). We first observed that uninephrectomized PIK3CA^{WT} mice treated with either vehicle or alpelisib had no particular phenotype, without proteinuria or kidney dysfunction (FIG. 2A, 2B, 2C). However, following surgery, PIK3CA^{Pod-HO} mice treated with vehicle rapidly developed a high amount of proteinuria with severe glomerular lesions (FIG. 2A, 2B, 2C, 2D). However, the amount of proteinuria significantly decreased in the uninephrectomized PIK3CA^{Pod-HO} mice treated with alpelisib (FIG. 2A and 2E). Consistently, compared to vehicle treated mice, PIK3CA^{Pod-HO} mice receiving alpelisib had better kidney function (FIG. 2B and 2C), preserved glomerular (FIG. 2D), fewer tubular dilation (data not shown), higher expression of podocytes markers (data not shown) and decreased podocyte AKT/mTOR pathway activation (data not shown). We concluded that alpelisib was able to prevent and to reverse disease in a mouse model carrying PIK3CA gain of function mutation in podocytes.

### Alpelisib Improves Different Mouse Models of Proliferative Glomerulonephritis

PIK3CA^{Pod} mouse models developed features of collapsing glomerulopathy and extracapillary disorders. In humans, collapsing glomerulopathy can be either primary or secondary to other diseases such as HIV or toxic exposures. Extracapillary disease is the hallmark of severe autoimmune disease affecting the kidneys such as lupus nephritis. Both types of glomerular lesions are associated with severe kidney injury and poor renal survival.

Crescentic glomerulonephritis is the hallmark of severe autoimmune disease affecting the kidneys such as lupus nephritis. Both types of glomerular lesions are associated with significant kidney damage and poor renal survival. To further investigate whether the AKT/mTOR pathway was activated in glomerular lesions of patients with these disorders, we conducted immunofluorescence experiments. These experiments confirmed the activation of the AKT/mTOR pathway specifically in glomerular epithelial cells, as compared to kidney biopsies from individuals with other diseases (data not shown). To compare the results obtained from single-cell RNA sequencing in the PIK3CA^{Pod-HE} mouse model, we further investigated the glomerular spatial transcriptomic changes in patients with extracapillary disorders using the GeoMx™ digital spatial profiler technology. Kidney biopsies from four patients with highly active lupus nephritis (LN) and four patients with minimal change disease (MCD) were selected for analysis (data not shown). A total of 49 glomeruli were selected as regions of interest (ROIs) (33 ROIs for LN and 16 ROIs for MCD). More than 18,000 genes were assayed in these ROIs (data not shown). The glomerular transcriptomes of LN samples showed distinct and broader clustering patterns compared to those of MCD samples (data not shown). Consistent with single cell RNA sequencing analysis in PIK3CA^{Pod-HET} mice, the gene expression profiles in LN glomeruli indicated a dedifferentiated state and elevated levels of inflammation-related transcripts (data not shown). We separately stained pAKT^{Ser473} in LN samples and classified each ROI based on the level of pAKT activity, with 20 ROIs classified as activity-high and 13 ROIs classified activity-low (data not shown). Higher pAKT activity correlated with gene set enrichment of WNT pathway, and significant upregulation of WNT4 (data not shown). Indeed, PIK3CA^{Po-HET} mutant mice demonstrate similar transcriptomic changes that closely resemble the patterns observed in LN patients.

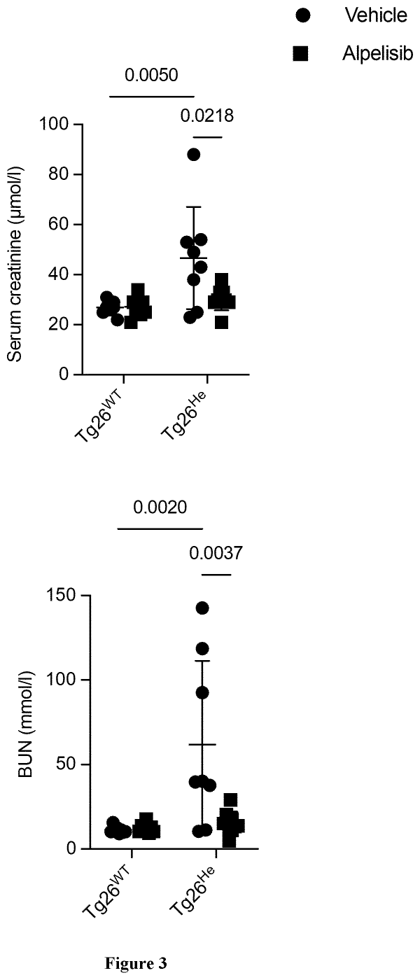

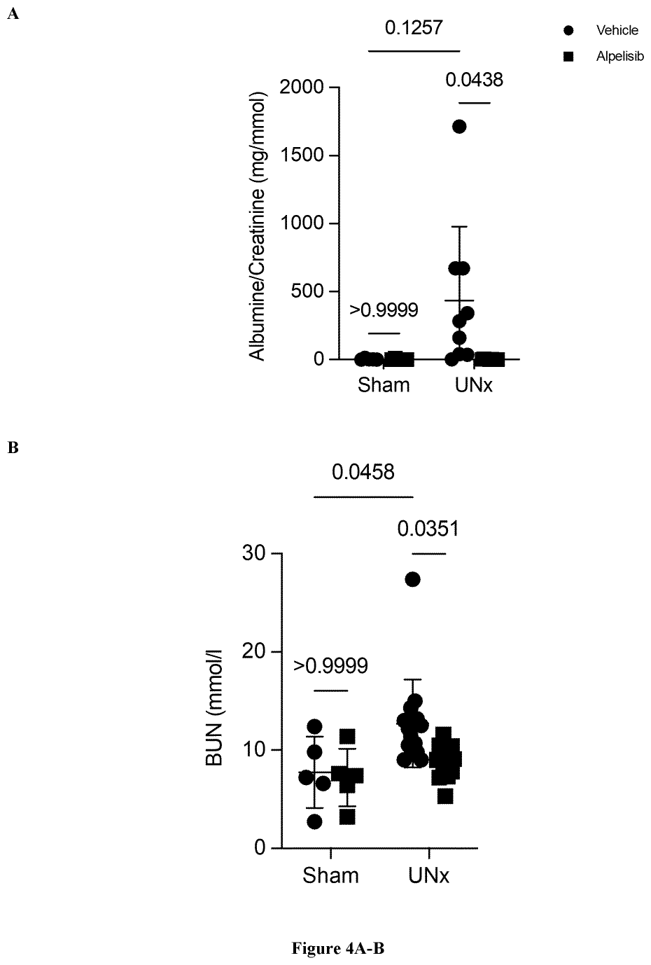

We wondered if alpelisib would be of interest in such glomerulonephritis where limited therapeutics are available. We started by exploring the degree of activation of the AKT/mTOR pathway in Tg26^{HE} mice, a model of collapsing glomerulopathy. This transgenic mouse model usually develops proteinuria by around 24 days of age with severe kidney lesions and die between 2 and 9 months. Four weeks old Tg26 mice (n=45) were randomly assigned to receive either vehicle (Tg26^{WT} n=9, Tg26^{HE} n=14) or alpelisib (Tg26^{WT} n=8, Tg26^{HE} n=14) during 8 weeks (data not shown). Tg26^{HE} mice treated with alpelisib demonstrated lower albuminuria (data not shown), a reduction in kidney volume (data not shown), less severe glomerular lesions (data not shown), higher expression of podocyte differentiation markers (data not shown), reduced expression in fibrotic markers, Col1a and Col3a (data not shown), and a lower expression of tubular injury markers, Lcn2 and KIM1 (data not shown), and an inflammatory marker, Tnfa (data not shown), compared to vehicle treated mice. Mechanistically, alpelisib was associated with profound AKT/mTOR pathway inhibition (data not shown). However, kidney function was not significantly improved (data not shown). The results were indeed encouraging but to overcome the variability of the model we decided to redo the experiment and to perform unilateral nephrectomy before treatment. This would accelerate kidney lesions and would allow to compare the paired kidneys. Four weeks old Tg26^{HE} and Tg26^{WT} mice underwent uninephrectomy and were then randomly assigned to vehicle (Tg26^{WT} n=8, Tg26^{HE} n=8) or alpelisib (Tg26^{WT} n=8, Tg26^{HE} n=9) treatment during 4 weeks (data not shown). At the time of sacrifice, we observed that whereas the surfaces of the kidneys of the uninephrectomized Tg26^{WT} or Tg26^{HE} treated with alpelisib were regular and smooth, the surfaces of the kidneys from Tg26^{HE} mice treated with vehicle were irregular (data not shown). Albuminuria was significantly improved in Tg26^{HE} mice treated with alpelisib compared to vehicle treated (data not shown). Ratio kidney/body weight was improved in uninephrectomized Tg26^{HE} mice treated with alpelisib (data not shown). Kidney histology showed that uninephrectomized Tg26^{HE} mice treated with alpelisib had fewer glomerular lesions (data not shown), maintained expression of podocyte markers (data not shown), reduced expression in Col1a, Col3a and Lcn2 (data not shown). AKT/mTOR pathway in glomeruli was profoundly blunted in mice treated with alpelisib (data not shown). Finally, kidney function was significantly improved in Tg26^{HE} mice treated with alpelisib compared to vehicle treated (FIG. 3).

To better appreciate the role of PIK3CA in podocytes during collapsing glomerulopathy, we decided to genetically remove PIK3CA specifically in these cells. To achieve this, we first interbreed Podocin Cre mice with PIK3CAlox/lox in order to generate PIK3CA^{Podo-KO}. These mice were then backcrossed with the FVB/N strain for 10 generation and interbreed with Tg26 mice in order to obtain Tg26^{PIK3CA-KO}. These mice were viable and had no particular phenotype at birth. With aging, Tg26^{PIK3CA-KO} developed less albuminuria, glomerular lesion, kidney fibrosis and improved kidney function compared to Tg26HE (data not shown). Indeed, PIK3CA deletion in podocytes is sufficient to reduce the intensity of collapsing glomerular lesions in Tg26^{HE} mouse model. Next, we sought to determine whether removing PIK3CA in podocytes later in life, when glomerular lesions were already established, could lead to an improvement in kidney lesions. For this purpose, we generated PIK3CA^{iPodo-KO} mice by backcrossing tamoxifen-inducible Podocin Cre mice with PIK3CA^{lox/lox} mice on the FVB/N strain (10 generations) and crossing them with Tg26^{HE} mice in order to obtain Tg26^{HE}PIK3CA^{iPodo-KO}. Cre recombination was induced in Tg26^{HE}PIK3CA^{iPodo-KO} mice at 3 weeks of age and the mice sacrificed at 12 weeks old. We observed that these mice exhibited improved glomerular lesion scores and reduced albuminuria compared to the control group (data not shown). Similarly, when Cre recombination was induced at 6 weeks of age, kidney lesions were reversed by the age of 12 weeks (data not shown). However, when Cre recombination was induced at later time point, specifically at 8 weeks of age, the progression of the disease was no longer affected, indicating that reversing the phenotype becomes challenging once the kidney lesions have become too severe (data not shown). In this model of collapsing glomerulopathy, we determined that the activation of PIK3CA in podocytes is of significant importance, and inhibiting it holds great promise as a potential therapeutic approach.

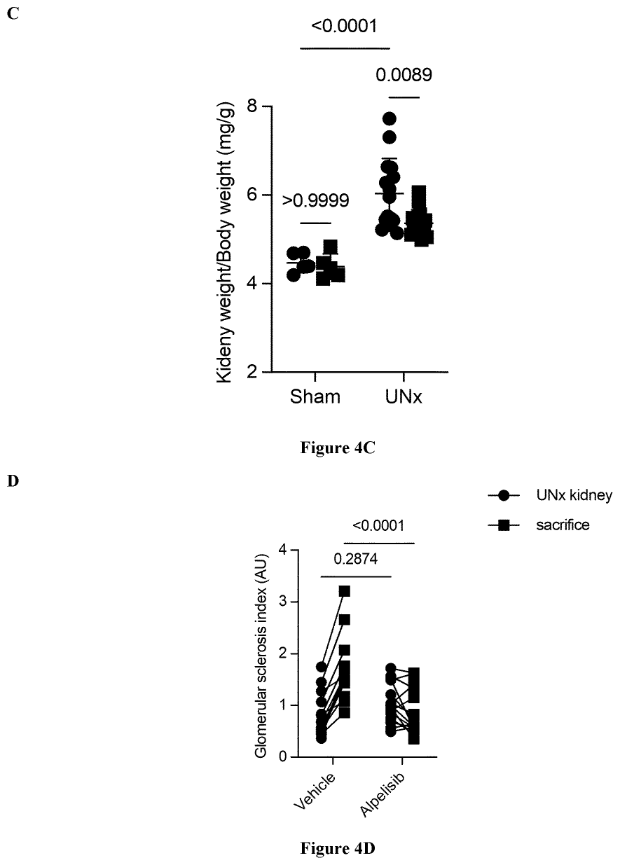

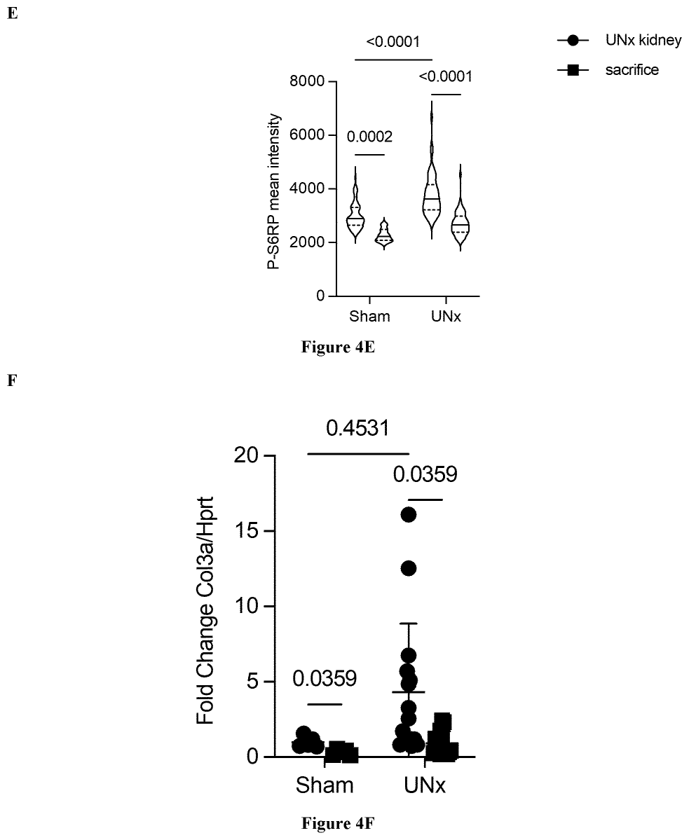

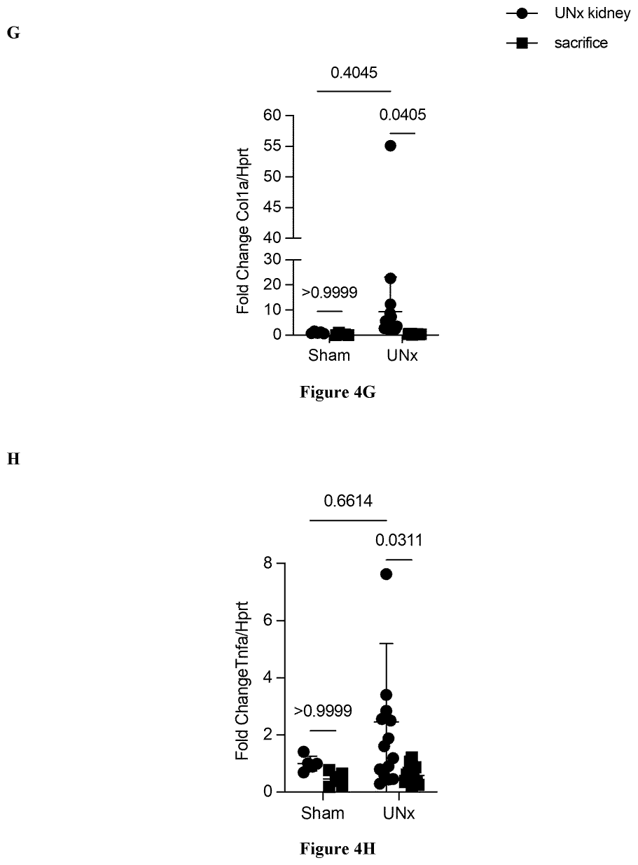

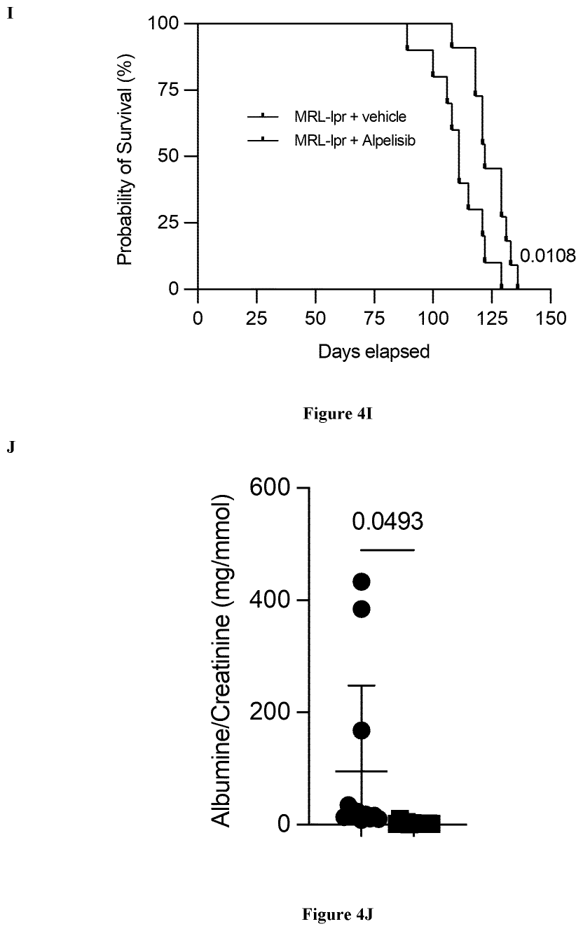

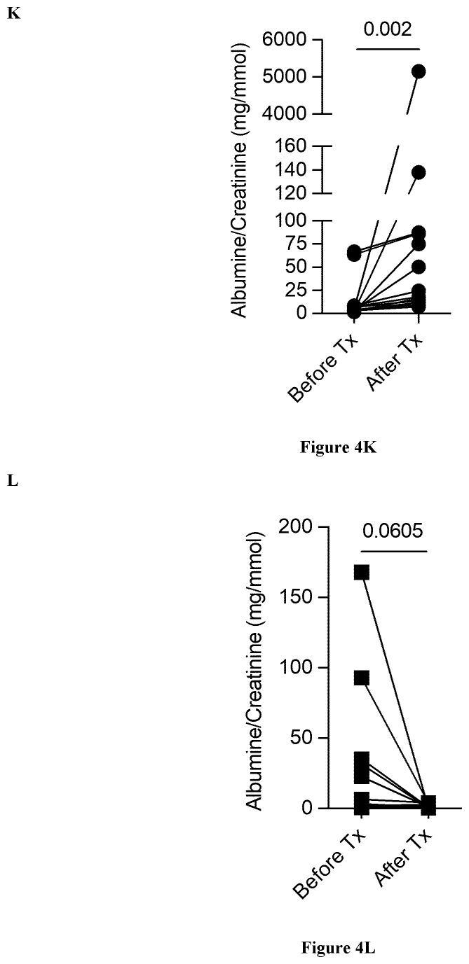

We next decided to investigate the impact of PIK3CA inhibition in extracapillary glomerulonephritis, particularly in lupus mice models. We first started with NZBWF1/J mice a well-established model of lupus like nephritis. NZBWF1/J mice have autoimmunity and develop progressive complex immune glomerulonephritis characterized by proteinuria and kidney dysfunction starting around the age of 25 weeks, but this model is characterized by its variability. In order to accelerate and uniformize the lesions, we performed uninephrectomy in 30 females aged of 24 weeks (data not shown). Of note, since the incidence and severity of symptoms are more pronounced in females and we used only females for our study. The mice were then randomly assigned to either vehicle (n=15) or alpelisib (n=15) during 4 weeks. At the end of the treatment period, the mice were sacrificed and their kidney histology was compared the one removed during uninephrectomy. At the time of uninephrectomy, there was no phenotypic difference including proteinuria and kidney histology between the two groups (data not shown). However, at the time of sacrifice, alpelisib treated mice demonstrated significantly less albuminuria (FIG. 4A) and lower blood urea nitrogen levels (FIG. 4B). Kidney to body weight ratio was significantly reduced in uninephrectomized NZBWF1/J mice receiving alpelisib (FIG. 4C). At the histological level, alpelisib treated mice had preserved glomeruli compared to vehicle (data not shown). Glomerular lesions severely progressed in vehicle treated uninephrectomized NZBWF1/mice, they remained stable in the alpelisib group (FIG. 4D). Glomerular AKT/mTOR pathway activation was blunted with alpelisib (FIG. 4D and 4E). Col1a and Col3a mRNA expression were reduced in mice treated with alpelisib (FIG. 4F and 4G) as well as TNFa (FIG. 4H). Podocyte differentiation markers were significantly improved in the group of uninephrectomized NZBWF1/J mice treated with alpelisib (data not shown). In NZBWF1/J mice treated with alpelisib, we observed partial correction of anemia and a significant increase in platelet count. Glomerular IgG, IgM and C3 deposits were accordingly significantly reduced in mice receiving alpelisib (data not shown). We then checked the level of dsDNA, an activity marker of SLE, and observed a tendency to decrease in NZBWF1/J mice treated with alpelisib (data not shown). Notably, spleen size was significantly reduced in both sham and uninephrectomized mice treated with alpelisib (data not shown).

We then decided to explore the relevance of alpelisib in MRL/MpJ-Faslpr/J mice (referred here as MRL-lpr), another mouse model of lupus nephritis. These mice with homozygous Fas mutation usually develop autoimmune disease resembling systemic lupus with lymphadenoproliferation, progressive renal failure and skin lesions. Female MRL-Ipr mice die at an average of 18-20 weeks old. We first randomly assigned to either vehicle or alpelisib MRL-lpr female mice at 8 weeks old, before they fully develop diseases. We confirmed prior drug administration that mice had already albuminuria. Alpelisib was associated with a significant extension of life expectancy (FIG. 4I). MRL-lpr mice treated with alpelisib demonstrated less proteinuria compared to vehicle (FIG. 4J). More stinkingly, comparison of albuminuria before and after treatment introduction showed opposite trajectories. Indeed, MRL-lpr mice treated with alpelisib demonstrated proteinuria improvement arguing for a reversibility of the disease (FIG. 4K and 4L). At sacrifice, MRL-lpr mice treated with alpelisib had a tendency to have a lower kidney to body weight ratio compared to vehicle treated animals (data not shown). Kidney examination showed that alpelisib was associated with no glomerular lesions compared to vehicle treated mice (data not shown) and an improved kidney function (data not shown).

dsDNA in MRL-lpr mice increases with time (data not shown). In order to further demonstrate the potential reversibility of the glomerular lesion with alpelisib we decided to randomly assign either to vehicle or alpelisib 12 weeks old MRL-lpr female mice. At this age, kidney lesions are already developed. Mice were then treated during 4 consecutive weeks (data not shown). Alpelisib was again associated with proteinuria reduction (data not shown). At the histological level, we found that glomerular lesion score was improved in the alpelisib group as well as a Cola1 and Cola3 mRNA expression (data not shown). Consistently glomerular AKT/mTOR pathway was inhibited in the group of alpelisib treated mice (data not shown). Expression of podocyte differentiation markers was increased in alpelisib mice compared to vehicle (data not shown). In addition, dsDNA antibodies, which were not different between the two groups at the start of the treatments, were significantly reduced with alpelisib treatment (data not shown).

We concluded that alpelisib and more generally PIK3CA inhibition represent promising drugs for patients with FSGS, lupus nephritis and more generally with proliferative glomerulonephritis.

### EXAMPLE 2

### Material and Methods

To accelerate and aggravate the renal lesions, uni-nephrectomies were performed (methodology described above) on NZB/NZBW F1 mice presenting lupus with renal damage. These mice were treated with different compounds: BYL719 (Alpelisib, Novartis), GDC-0077 (inavolisib, Genentech/Roche), TAK-117/MLN1117/INK1117 (serabelisib), GDC-0032 (Taselisib, Genentech/Roche) or rapamycin.

### Results

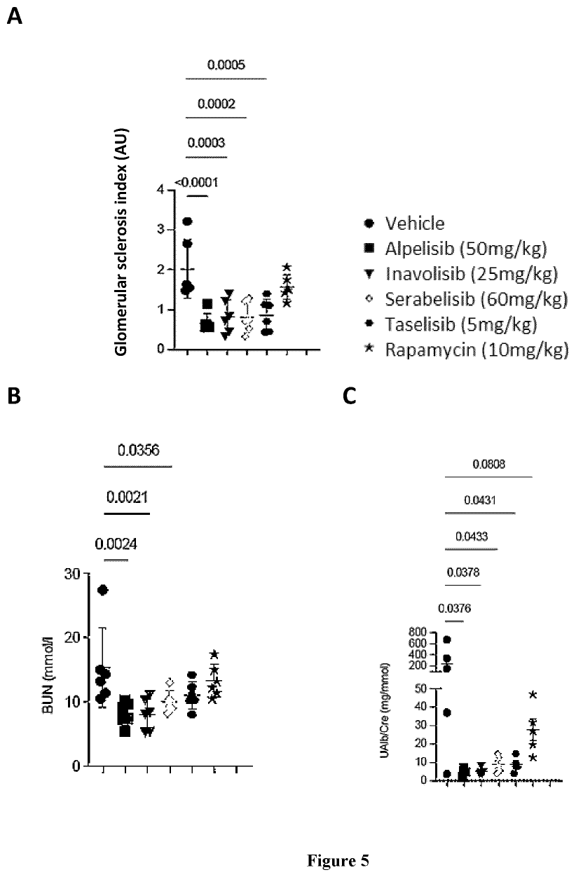

The results are depicted in FIGS. 5A, 5B, 5C and demonstrate that PIK3CA inhibitors represents promising drugs for patients with proliferative glomerulonephritis.

### REFERENCES

Throughout this application, various references describe the state of the art to which this invention pertains. The disclosures of these references are hereby incorporated by reference into the present disclosure.
1 Bilanges, B., Posor, Y. & Vanhaesebroeck, B. PI3K isoforms in cell signalling and vesicle trafficking. Nat Rev Mol Cell Biol, doi: 10.1038/s41580-019-0129-z (2019).
2 Welch, H. C., Coadwell, W. J., Stephens, L. R. & Hawkins, P. T. Phosphoinositide 3-kinase-dependent activation of Rac. FEBS Lett 546, 93-97 (2003).
3 Kurek, K. C. et al. Somatic mosaic activating mutations in PIK3CA cause CLOVES syndrome. Am J Hum Genet 90, 1108-1115, doi: 10.1016/j.ajhg.2012.05.006 (2012).
4 Keppler-Noreuil, K. M. et al. Clinical delineation and natural history of the PIK3CA-related overgrowth spectrum. Am J Med Genet A 164A, 1713-1733, doi: 10.1002/ajmg.a.36552 (2014).
5 Keppler-Noreuil, K. M. et al. PIK3CA-related overgrowth spectrum (PROS): diagnostic and testing eligibility criteria, differential diagnosis, and evaluation. Am J Med Genet A 167A, 287-295, doi: 10.1002/ajmg.a.36836 (2015).
6 Luks, V. L. et al. Lymphatic and other vascular malformative/overgrowth disorders are caused by somatic mutations in PIK3CA. J Pediatr 166, 1048-1054 e1041-1045, doi: 10.1016/j.jpeds.2014.12.069 (2015).
7 Mirzaa, G. M. et al. Megalencephaly-capillary malformation (MCAP) and megalencephaly-polydactyly-polymicrogyria-hydrocephalus (MPPH) syndromes: two closely related disorders of brain overgrowth and abnormal brain and body morphogenesis. Am J Med Genet A 158A, 269-291, doi: 10.1002/ajmg.a.34402 (2012).
8 Rios, J. J. et al. Somatic gain-of-function mutations in PIK3CA in patients with macrodactyly. Hum Mol Genet 22, 444-451, doi: 10.1093/hmg/dds440 (2013).
9 Rivière, J. B. et al. De novo germline and postzygotic mutations in AKT3, PIK3R2 and PIK3CA cause a spectrum of related megalencephaly syndromes. Nat Genet 44, 934-940, doi: 10.1038/ng.2331 (2012).
10 Canaud, G. et al. AKT2 is essential to maintain podocyte viability and function during chronic kidney disease. Nat Med 19, 1288-1296, doi: 10.1038/nm.3313 (2013).
11 Muzumdar, M. D., Tasic, B., Miyamichi, K., Li, L. & Luo, L. A global double-fluorescent Cre reporter mouse. Genesis 45, 593-605, doi: 10.1002/dvg.20335 (2007).

1. A method for treating proliferative glomerulonephritis in a subject in need thereof comprising administering to the subject a therapeutically effective amount of a PIK3CA inhibitor.

2. The method according to claim 1, wherein the method consists essentially in a step of administrating to the subject a therapeutically effective amount of a PIK3CA inhibitor.

3. The method according to claim 1, wherein the proliferative glomerulonephritis is an infectious disease, or a multisysteme disease.

4. The method according to claim 1, wherein the proliferative glomerulonephritis is lupus nephritis.

5. The method according to claim 1, wherein the proliferative glomerulonephritis is focal and segmental glomerulosclerosis.

6. The method according to claim 1, wherein the PIK3CA inhibitor is selected from the group consisting of: BYL719 (Alpelisib), A66, GDC-0077 (inavolisib), CYH33 (risovalisib), and TAK-117/MLN1117/INK1117 (serabelisib) and pharmaceutically acceptable salts thereof.

7. (canceled)

8. The method according to claim 1, wherein the PIK3CA inhibitor is BYL719 or a derivatives thereof.

9. The method of claim 1, wherein the PIK3CA inhibitor is administered simultaneously, separately or sequentially with a classical treatment of proliferative glomerulonephritis.

10. The method according to claim 9, wherein the classical treatment is selected from the group consisting of: an immunosuppressor, glucocorticoids, a MAPK inhibitor, a PAK inhibitor, an mTOR inhibitor, a TKI inhibitor, a PARP inhibitor and/or an EGFR inhibitors.

11. (canceled)

12. (canceled)

13. A pharmaceutical composition comprising a PIK3CA inhibitor and a classical treatment as a combined preparation wherein the pharmaceutical composition is formulated for simultaneous, separate or sequential use in the treatment of proliferative glomerulonephritis.

14. A method of screening an inhibitor of PIK3CA comprising i) providing a test compound and ii) determining the ability of said test compound to inhibit and/or reduce the activity and/or expression of PIK3CA.

15. The method according to claim 3, wherein the infectious disease is selected from the group consisting of poststreptococcal glomerulonephritis, infective endocarditis, occult visceral sepsis, hepatitis B infection with vasculitis and/or cryoglobulinemia, HIV infection, hepatitis C with cryoglobulinemia and membranoproliferative glomerulonephritis; and wherein the multisystem disease is selected from the group consisting of systemic lupus erythematosus, IgA nephropathy, Henoch-Schönlein purpura, systemic necrotizing vasculitis, granulomatosis with polyangiitis type Wegener, Goodpasture's syndrome, essential mixed cryoglobulinemia, malignancy, relapsing polychondritis and rheumatoid arthritis with vasculitis.
